SEQUENCE LISTING (33 sequences)

SEQ ID NO: 1 - DNA, 1320 bp, mus musculus
atggcctctctgaagaggtttcagacgctcgtgcccctggatcacaaacaaggtaccttatttgaaattattggagagcccaagttgcccaagtggttccatgtcgaatgcctggaagatccaaaaagactgtacgtggaacctcggctactggaaatcatgtttggtaaggatggagagcacatcccacatcttgaatctatgttgcacaccctgatacatgtgaacgtgtggggccctgaaaggcgagctgagatttggatattcggaccgccgcctttccgaagggacgttgaccggatgctcactgatctggctcactattgccgcatgaaactgatggaaatagaggctctggaggctggagttgagcgtcgtcgtatggcggcccataaggctgccacccagcctgctcccgtgaaggtccgcgaggctgcccctcggcccgcttccgtgaaggtccctgagacggccacccagcctgctcccgtgaaggtccgcgaggctgcccctcagcccgctccggtgcaggaggtccgcgaggctgcccctcagcaggcttccgtgcaggaggaggtccgcgaggctgccaccgagcaggctcccgtgcaggaggtccgcgaggctgccaccgagcaggctcccgtgcaggaggtcagcgaggctgccaccgagcaggctcccgtgcaggaggtcaacgaggctgccaccgagcaggcttccgtgcaggcggtccgcgaggctgccacccggccggctcccgggaaggtccgcaaggcggccacccagccggctccggtgcaggtttgccaggaggccacccagttggctcccgtgaaggtccgcgaggcggccacccagccggcttccgggaaggtccgcgaggcggccacccagttggctcctgtgaaggtccgcaaggcagccacccagttggctcctgtgaaggtccacgaggcggccacccagccggctccggggaaggtcagcgatgctgccacgcagtcggcttcggtgcaggttcgtgaggctgccacgcagctgtctcccgtggaggccactgatactagccagttggctcaggtgaaggctgatgaagcctttgcccagcacacttcaggggaggcccaccaggttgccaatgggcagtctcccattgaagtctgtgagactgccaccgggcagcattctctagatgtctctagggccttgtcccagaagtgtcctgaggtttttgagtgggagacccagagttgtttggatggcagctatgtcatagttcagcctccaagggatgcctgggaatcatttatcatatta

SEQ ID NO: 2 - protein, 440 aa, mus musculus
MASLKRFQTLVPLDHKQGTLFEIIGEPKLPKWFHVECLEDPKRLYVEPRLLEIMFGKDGEHIPHLESMLHTLIHVNVWGPERRAEIWIFGPPPFRRDVDRMLTDLAHYCRMKLMEIEALEAGVERRRMAAHKAATQPAPVKVREAAPRPASVKVPETATQPAPVKVREAAPQPAPVQEVREAAPQQASVQEEVREAATEQAPVQEVREAATEQAPVQEVSEAATEQAPVQEVNEAATEQASVQAVREAATRPAPGKVRKAATQPAPVQVCQEATQLAPVKVREAATQPASGKVREAATQLAPVKVRKAATQLAPVKVHEAATQPAPGKVSDAATQSASVQVREAATQLSPVEATDTSQLAQVKADEAFAQHTSGEAHQVANGQSPIEVCETATGQHSLDVSRALSQKCPEVFEWETQSCLDGSYVIVQPPRDAWESFIIL

SEQ ID NO: 3 - DNA, 1248 bp, mus musculus
atggcctctctgaagaggtttcagacgctcgtgcccctggatcacaaacaaggtaccttatttgaaattattggagagcccaagttgcccaagtggttccatgtcgaatgcctggaagatccaaaaagactgtacgtggaacctcggctactggaaatcatgtttggtaaggatggagagcacatcccacatcttgaatctatgttgcacaccctgatacatgtgaacgtgtggggccctgaaaggcgagctgagatttggatattcggaccgccgcctttccgaagggacgttgaccggatgctcactgatctggctcactattgccgcatgaaactgatggaaatagaggctctggaggctggagttgagcgtcgtcgtatggcggcccataaggctgccacccagcctgctcccgtgaaggtccgcgaggctgcccctcagcccgctccggtgcaggaggtccgcgaggctgcccctcagcaggcttccgtgcaggaggaggtccgcgaggctgccaccgagcaggctcccgtgcaggaggtccgcgaggctgccaccgagcaggctcccgtgcaggaggtcagcgaggctgccaccgagcaggctcccgtgcaggaggtcaacgaggctgccaccgagcaggcttccgtgcaggcggtccgcgaggctgccacccggccggctcccgggaaggtccgcaaggcggccacccagccggctccggtgcaggtttgccaggaggccacccagttggctcccgtgaaggtccgcgaggcggccacccagccggcttccgggaaggtccgcgaggcggccacccagttggctcctgtgaaggtccgcaaggcagccacccagttggctcctgtgaaggtccacgaggcggccacccagccggctccggggaaggtcagcgatgctgccacgcagtcggcttcggtgcaggttcgtgaggctgccacgcagctgtctcccgtggaggccactgatactagccagttggctcaggtgaaggctgatgaagcctttgcccagcacacttcaggggaggcccaccaggttgccaatgggcagtctcccattgaagtctgtgagactgccaccgggcagcattctctagatgtctctagggccttgtcccagaagtgtcctgaggtttttgagtgggagacccagagttgtttggatggcagctatgtcatagttcagcctccaagggatgcctgggaatcatttatcatatta

SEQ ID NO: 4 - protein, 416 aa, mus musculus
MASLKRFQTLVPLDHKQGTLFEIIGEPKLPKWFHVECLEDPKRLYVEPRLLEIMFGKDGEHIPHLESMLHTLIHVNVWGPERRAEIWIFGPPPFRRDVDRMLTDLAHYCRMKLMEIEALEAGVERRRMAAHKAATQPAPVKVREAAPQPAPVQEVREAAPQQASVQEEVREAATEQAPVQEVREAATEQAPVQEVSEAATEQAPVQEVNEAATEQASVQAVREAATRPAPGKVRKAATQPAPVQVCQEATQLAPVKVREAATQPASGKVREAATQLAPVKVRKAATQLAPVKVHEAATQPAPGKVSDAATQSASVQVREAATQLSPVEATDTSQLAQVKADEAFAQHTSGEAHQVANGQSPIEVCETATGQHSLDVSRALSQKCPEVFEWETQSCLDGSYVIVQPPRDAWESFIIL

SEQ ID NO: 5 - DNA, 1242 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctggtagaaggggacattatccggccaagccctttccgattgttgtcagtgaccaataataaatggcccaagggcgttggtggctttgtggagatccccttcctgctttccagaaagtatgatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 6 - protein, 414 aa, mus musculus
MGAPSASRCSGVCSTSVPEGFTPEGSPVFQDKDIPAINQGLISEETPESSFLVEGDIIRPSPFRLLSVTNNKWPKGVGGFVEIPFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 7 - DNA, 1176 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctggtagaaggggacattatccggccaggggtcagccacggtgtgtctttcccagatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 8 - protein, 392 aa, mus musculus
MGAPSASRCSGVCSTSVPEGFTPEGSPVFQDKDIPAINQGLISEETPESSFLVEGDIIRPGVSHGVSFPDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKPGPGAGADSLSLEQFQLAQAPTVPLALFPEARDKPAPIQDAFERLAPLPGGCAPGSHIREVPRD

SEQ ID NO: 9 - DNA, 1140 bp, mus musculus
atgggagcaccctcagcatccagatgttctggagtctgcagtaccagtgttccagaaggcttcactcctgagggaagcccggtatttcaggacaaggacatccccgcaattaaccaagggctcatctcagaggagaccccagaaagcagcttcctgctttccagaaagtatgatgaactcagccgccgggtcattatggatgcctttgctgagtttgaacgtttcacatgcatccggtttgttgcctaccatggtcagagagactttgtttccattcttcctatggcggggtgtttctctggtgtgggacgcagtggagggatgcaggtggtgtccttggcacccacttgtctccggaagggccgaggcattgtcctacatgagctcatgcacgtacttggcttctggcatgagcattcacgggcagatcgggaccgctacatccaagtcaactggaacgagatcctcccgggctttgaaatcaacttcatcaagtcacggagtaccaatatgttagttccctatgactactcatctgtgatgcattatgggagatttgccttcagctggcgtgggcagcccaccatcataccactctggacctccagtgttcacattggccagcgatggaacctgagtacctcagatatcacccgggtctgcaggctgtataactgcagccggagtgtccctgactcccacgggagagggtttgaggcccagagtgatggaagcagcctcacccctgcctctatatcacgtctacaaagacttctcgaggcactgtcagaggaatctggaagctctgcccctagtggctccaggactggaggccagagtattgccgggcttggtaacagccagcaaggatgggagcatcctcctcagagcacattcagtgtgggagccttggcaagaccacctcagatgctagccgatgcttcaaaatcggggcctggagcaggtgcagacagcttgtctctagagcagttccagctagcccaggcccccactgtacctcttgctctatttccagaagccagagacaagccagcacctatccaagatgcctttgagaggctagctccacttccaggaggctgtgcacctggaagtcacattagagaggtgcccagagac

SEQ ID NO: 10 - protein, 380 aa, Mus musculus
MGAPSASRCSGVCSTSVPEGFTPEGSPVFQDKDIPAINQGLISEETPESSFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 11 - DNA, 384 bp, mus musculus
aaggtcttcagtcgctgtgagctggccaaagagatgcatgacttcggtctggatggctaccggggttataacctggctgactgggtctgccttgcttactacacaagtggcttcaacacaaatgctgtggatcatgaagctgatggaagcaccaacaatggcatcttccagatcagcagccggaggtggtgcagaaccctcgcctcgaatggccccaatctttgcaggatatactgcactgatttgttgaacaatgatctcaaagattctatcgtctgtgccatgaagatagttcaagaacccctgggtctgggctattgggaagcctggaggcaccactgccagggcagggacctcagtgactgggtggatggctgtgacttc

SEQ ID NO: 12 - protein, 128 aa, mus musculus
KVFSRCELAKEMHDFGLDGYRGYNLADWVCLAYYTSGFNTNAVDHEADGSTNNGIFQISSRRWCRTLASNGPNLCRIYCTDLLNNDLKDSIVCAMKIVQEPLGLGYWEAWRHHCQGRDLSDWVDGCDF

SEQ ID NO: 13 - DNA, 820 bp, mus musculus
gccctggcaaggttgtgggggacatcttgagctgaagcagggttttgagccactgctgctgctgccattgtcaccatggtctcagctctgcggggagcacccctgatcagggtgcactcaagccctgtttcttctccttctgtgagtggaccacggaggctggtgagctgcctgtcatcccaaagctcagctctgagccagagtggtggtggctccacctctgccgccggcatagaagccaggagcagggctctcagaaggcggtggtgcccagctgggatcatgttgttggccctggtctgtctgctcagctgcctgctaccctccagtgaggccaagctctacggtcgttgtgaactggccagagtgctacatgacttcgggctggacggataccggggatacagcctggctgactgggtctgccttgcttatttcacaagcggtttcaacgcagctgctttggactacgaggctgatgggagcaccaacaacgggatcttccagatcaacagccggaggtggtgcagcaacctcaccccgaacgtccccaacgtgtgccggatgtactgctcagatttgttgaatcctaatctcaaggataccgttatctgtgccatgaagataacccaagagcctcagggtctgggttactgggaggcctggaggcatcactgccagggaaaagacctcactgaatgggtggatggctgtgacttctaggatggacggaaccatgcacagcaggctgggaaatgtggtttggttcctgacctaggcttgggaagacaagccagcgaataaaggatggttgaacgtt

SEQ ID NO: 14 - protein, 215 aa, homo sapien [sequence omitted]

SEQ ID NO: 15 - DNA, 417 bp, mus musculus
aagatttatgaacgctgtgagctggcaaagaagctggaggaggctggcctcgatggcttcaaaggctatactgttggagactggctgtgtgtggcacactatgagagtggctttgacacctcttttgtggaccacaatccagatggcagcagtgaatatggcattttccagctgaactctgcctggtggtgtaacaatggcatcacacccactcagaacctctgcaacatcgattgtaatgacctgctcaaccgccatattctggatgatatcatatgtgccaagagggttgcatcctcacataagagtatgaaggcctgggattcctggacccagcactgtgccggtcatgatttatcagaatggctaaaggggtgttctgtgcgtctgaaaactgactcaagctataataactgg

SEQ ID NO: 16 - protein, 139 aa, mus musculus
KIYERCELAKKLEEAGLDGFKGYTVGDWLCVAHYESGFDTSFVDHNPDGSSEYGIFQLNSAWWCNNGITPTQNLCNIDCNDLLNRHILDDIICAKRVASSHKSMKAWDSWTQHCAGHDLSEWLKGCSVRLKTDSSYNNW

SEQ ID NO: 17 - DNA, 588 bp, homo sapien
ctgggagggcttacaggtgccataatgaaggcctggggcactgtggtagtgaccttggccacgctgatggttgtcactgtggatgccaagatctatgaacgctgcgagctggcggcaagactggagagagcagggctgaacggctacaagggctacggcgttggagactggctgtgcatggctcattatgagagtggctttgacaccgccttcgtggaccacaatcctgatggcagcagtgaatatggcattttccaactgaattctgcctggtggtgtgacaatggcattacacccaccaagaacctctgccacatggattgtcatgacctgctcaatcgccatattctggatgacatcaggtgtgccaagcagattgtgtcctcacagaatgggctttctgcctggacttcttggaggctacactgttctggccatgatttatctgaatggctcaaggggtgtgatatgcatgtgaaaattgatccaaaaattcatccatgactcagattcgaagagacagattttatcttcctttcatttctttctcttgtgcatttaataaaggatggtatctataaacaatgc

SEQ ID NO: 18 - protein, 159 aa, homo sapien
MKAWGTVVVTLATLMVVTVDAKIYERCELAARLERAGLNGYKGYGVGDWLCMAHYESGFDTAFVDHNPDGSSEYGIFQLNSAWWCDNGITPTKNLCHMDCHDLLNRHILDDIRCAKQIVSSQNGLSAWTSWRLHCSGHDLSEWLKGCDMHVKIDPKIHP

SEQ ID NO: 19 - protein, 435 aa, mus musculus
MGIMGSLWPWILTMLSLLGLSMGAPSASRCSGVCSTSVPEGFTPEGSPVFQDKDIPAINQGLISEETPESSFLVEGDIIRPSPFRLLSVTNNKWPKGVGGFVEIPFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 20 - protein, 401 aa, mus musculus
MGIMGSLWPWILTMLSLLGLSMGAPSASRCSGVCSTSVPEGFTPEGSPVFQDKDIPAINQGLISEETPESSFLLSRKYDELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 21 - protein, 413 aa, mus musculus
MGIMGSLWPWILTMLSLLGLSMGAPSASRCSGVCSTSVPEGFTPEGSPVFQDKDIPAINQGLISEETPESSFLVEGDIIRPGVSHGVSFPNELSRRVIMDAFAEFERFTCIRFVAYHGQRDFVSILPMAGCFSGVGRSGGMQVVSLAPTCLRKGRGIVLHELMHVLGFWHEHSRADRDRYIQVNWNEILPGFEINFIKSRSTNMLVPYDYSSVMHYGRFAFSWRGQPTIIPLWTSSVHIGQRWNLSTSDITRVCRLYNCSRSVPDSHGRGFEAQSDGSSLTPASISRLQRLLEALSEESGSSAPSGSRTGGQSIAGLGNSQQGWEHPPQSTFSVGALARPPQMLADASKSGPGAGADSLSLEQFQLAQAPTVPLALFPEARDKPAPIQDAFERLAPLPGGCAPGSHIREVPRD

SEQ ID NO: 22 - DNA, 1296 bp, homo sapiens
atggagggtgtagggggtctctggccttgggtgctgggtctgctctccttgccaggtgtgatcctaggagcgcccctggcctccagctgcgcaggagcctgtggtaccagcttcccagatggcctcacccctgagggaacccaggcctccggggacaaggacattcctgcaattaaccaagggctcatcctggaagaaaccccagagagcagcttcctcatcgagggggacatcatccggccgagtcccttccgactgctgtcagcaaccagcaacaaatggcccatgggtggtagtggtgtcgtggaggtccccttcctgctctccagcaagtacgatgagcccagccgccaggtcatcctggaggctcttgcggagtttgaacgttccacgtgcatcaggtttgtcacctatcaggaccagagagacttcatttccatcatccccatgtatgggtgcttctcgagtgtggggcgcagtggagggatgcaggtggtctccctggcgcccacgtgtctccagaagggccggggcattgtccttcatgagctcatgcatgtgctgggcttctggcacgagcacacgcgggccgaccgggaccgctatatccgtgtcaactggaacgagatcctgccaggctttgaaatcaacttcatcaagtctcagagcagcaacatgctgacgccctatgactactcctctgtgatgcactatgggaggctcgccttcagccggcgtgggctgcccaccatcacaccactttgggcccccagtgtccacatcggccagcgatggaacctgagtgcctcggacatcacccgggtcctcaaactctacggctgcagcccaagtggccccaggccccgtgggagagggtcccatgcccacagcactggtaggagccccgctccggcctccctatctctgcagcggcttttggaggcactgtcggcggaatccaggagccccgaccccagtggttccagtgcgggaggccagcccgttcctgcagggcctggggagagcccacatgggtgggagtcccctgccctgaaaaagctcagtgcagaggcctcggcaaggcagcctcagaccctagcttcctccccaagatcaaggcctggagcaggtgcccccggtgttgctcaggagcagtcctggctggccggagtgtccaccaagcccacagtcccatcttcagaagcaggaatccagccagtccctgtccagggaagcccagctctgccagggggctgtgtacctagaaatcatttcaaggggatgtccgaagattaa

SEQ ID NO: 23 - protein, 431 aa, homo sapiens
MEGVGGLWPWVLGLLSLPGVILGAPLASSCAGACGTSFPDGLTPEGTQASGDKDIPAINQGLILEETPESSFLIEGDIIRPSPFRLLSATSNKWPMGGSGVVEVPFLLSSKYDEPSRQVILEALAEFERSTCIRFVTYQDQRDFISIIPMYGCFSSVGRSGGMQVVSLAPTCLQKGRGIVLHELMHVLGFWHEHTRADRDRYIRVNWNEILPGFEINFIKSQSSNMLTPYDYSSVMHYGRLAFSRRGLPTITPLWAPSVHIGQRWNLSASDITRVLKLYGCSPSGPRPRGRGSHAHSTGRSPAPASLSLQRLLEALSAESRSPDPSGSSAGGQPVPAGPGESPHGWESPALKKLSAEASARQPQTLASSPRSRPGAGAPGVAQEQSWLAGVSTKPTVPSSEAGIQPVPVQGSPALPGGCVPRNHFKGMSED

SEQ ID NO: 24 - protein, 11 aa, mus musculus
HELMHVLGFWH

SEQ ID NO: 25 - protein, 5 aa, mus musculus
SVMHY

SEQ ID NO: 26 - protein, 11 aa, mus musculus
  MISC_FEATURE: The X represents possible amino acid residue substitution with other amino acids
HEXXHXXGXXH

SEQ ID NO: 27 - protein, 5 aa, mus musculus
  UNSURE (1)..(5): X indicates the site of possible amino acid substitution
SXMHY

SEQ ID NO: 28 - DNA, 24 bp, Homo sapiens
gcgcccctggcctccagctgcgca

SEQ ID NO: 29 - DNA, 24 bp, homo sapiens
cacgacaccactaccacccatggg

SEQ ID NO: 30 - DNA, 24 bp, homo sapiens
ggctgcagcccaagtggccccagg

SEQ ID NO: 31 - DNA, 24 bp, homo sapiens
agcaacaccgggggcacctgctcc

SEQ ID NO: 32 - DNA, 24 bp, homo sapiens
gaggtccccttcctgctctccagc

SEQ ID NO: 33 - DNA, 24 bp, homo sapiens
ggcatgggaccctctcccacgggg

(19) United States
(12) Patent Application Publication
(10) Pub. No.: US 2015/0125445 A1
(43) Pub. Date: May 7, 2015

(54) COMPOSITIONS AND METHODS FOR TREATING CANCER

(71) Applicant: University of Virginia Patent Foundation, d/b/a University of Virginia Licensing & Ventures Group, Charlottesville, VA (US)

(72) Inventors: John C. Herr, Charlottesville, VA (US); Eusebio S. Pires, Charlottesville, VA (US); Austin Herr, Charlottesville, VA (US)

(21) Appl. No.: 14/407,399

(22) PCT Filed: Jun. 13, 2013
(86) PCT No.: PCT/US13/45745

§ 371 Date: Dec. 11, 2014

Related U.S. Application Data
(60) Provisional application No. 61/659,049, filed on Jun. 13, 2012.

(51) Int. Cl. A61K 47/48; G01N 33/574

(52) U.S. Cl. CPC A61K 47/48469; G01N 33/57492; C07K 2317/24; C07K 2317/54; C07K 2317/50; C07K 2317/55; C07K 2319/55; C07K 2317/21; C07K 2317/74; C12Y 302/02022; C07K 2317/732
USPC 424/133.1; 424/183.1; 424/139.1; 435/188; 435/7.23; 435/6.14; 435/6.12; 435/6.11

(57) ABSTRACT

The present invention provides compositions for targeting SAS1B positive cancer cells using immunotoxin technology and discloses that kidney and pancreatic cancer cells are SAS1B positive, but not normal kidney and pancreatic cells. The invention discloses that despite being expressed only in growing oocytes in females among normal tissues SAS1B is expressed in cancers of both men and women.

### CROSS REFERENCE TO RELATED APPLICATIONS

This application is entitled to priority pursuant to 35 U.S.C. 119(e) to U.S. Provisional Patent Application Ser. No. 61/659,049, filed Jun. 13, 2012, the entire disclosure of which is herein incorporated by reference.

### STATEMENT REGARDING FEDERALLY SPONSORED RESEARCH OR DEVELOPMENT

This invention was made in part with United States Government support under Grant Nos. D43TW/HD00654 awarded by Fogarty International Center at the NIH. The United States Government has certain rights in the invention.

### BACKGROUND

The astacin family of metalloendopeptidases was recognized as a novel family of proteases in the 1990s. The astacins are a subfamily of the metzincin superfamily of metalloproteinases. The first to be characterized was the crayfish enzyme astacin. To date more than 200 members of this family have been identified in species ranging from bacteria to humans. Astacins are involved in developmental morphogenesis, matrix assembly, tissue differentiation, and digestion. Family members include the procollagen C-proteinase (BMP1, bone morphogenetic protein 1), tolloid and mammalian tolloid-like, HMP (Hydra vulgaris metalloproteinase), sea urchin BP 10 (blastula protein) and SPAN (Strongylocentrotus purpuratus astacin), the ‘hatching’ subfamily comprising alveolin, ovastacin, LCE, HCE (′low′ and ‘high’ choriolytic enzymes), nephrosin (from carp head kidney), UVS.2 from frog, and the meprins. In the human and mouse genomes, there are six astacin family genes (two meprins, three BMP1/tolloid-like, one ovastacin), but in Caenorhabditis elegans there are 40.

Astacin family members are characterized by a unique 18-amino acid signature sequence, which begins with a five-amino acid zinc-binding motif found in most metalloendopeptidases (See review by Bond and Benyon, 1995). The signature sequence is part of an approximately 200-amino acid sequence, which are the entire mature crayfish astacin and the catalytic or protease domain of all the members of the family. Signal and prosequences are also common features of family members, with the possible exception of QuCAM-1; these NH2-terminal domains have yet to be found for this latter protein. Astacin, which until recently was only studied as a mature protein that begins with the protease domain, is now known to contain a prepro segment of 49 residues. The transient signal peptides direct the proteins into the endoplasmic reticulum during biosynthesis, which is consistent with the finding that all of the proteins of the family studied thus far are secreted or plasma membrane bound. The prosequences vary greatly in size, containing up to 519 amino acids for Drosophila tolloid-related-1 (DrT/r-1), and are likely to be important for regulating activity and perhaps expression of the proteases. Regarding the latter point, for example, the large prosequence of DrT/r-1 has been suggested to prevent expression of this gene product in early stages of embryogenesis when cell cycles are very short.

Meprins are zinc-dependent, membrane-bound proteases and members of the “astacin family” of metalloproteinases (Bond and Beynon, 1995, Protein Sci. 4: 1247-1261; Sterchi et al., 2008, Molecular Aspects of Medicine, 29:309-328). The enzymes are multidomain, oligomeric proteins. The expression is highly regulated on the transcriptional and translational level. Typically, the proteins are targeted to apical membranes of polarized epithelial cells (Eldering et al., Eur. J. Biochem. 247: 920-932, 1997). Various growth factors, cytokines, and extracellular matrix proteins are substrates for meprins. Meprins have been identified in leukocytes, cancer cells and intestine and kidney. Both the meprin α and β genes are expressed in various cancer cells. In colorectal tumour tissue meprin α mRNA, immunoreactive protein and enzymatic activity is detected. In contrast to normal colon, however, the meprin a subunit is secreted into the stroma of the tumor where it accumulates and can be detected by immuno-histochemical methods. The mechanism of this aberrant secretion was shown using a colon adenocarcinoma cell line (Caco-2) expressing meprin a endogenously. When cultured on transwell filter supports meprin is equally secreted from the apical and the basolateral membrane domains. On the basolateral side of the epithelial cell layer, meprin a may be activated by plasmin, which is generated from plasminogen by an activation process catalyzed by uPA from intestinal fibroblasts (Rösmann et al., 2002). Meprin expression may play a role in tumor cell invasion and migration and in doing so may be involved in tumor progression (Sterchi et al., 2008; Rosmann et al., 2002, J. Biol. Chem., 277:43:40650-40658).

Quesada et al. (2004, J. Biol. Chem., 279:25:26627-26634) isolated a novel protein from mouse and human and because of its predominant expression in ovarian tissues and apparent similarity to astacins named it “ovastacin”. Quesada was looking for candidate metalloproteinases involved in the process of embryo hatching and used the BLAST algorithm to look for novel astacin metalloproteinases. They discovered and sequenced a novel protein in mouse and human and localized the gene in humans to human chromosome 2q11.1. Computer analysis revealed an N-terminal signal peptide, a zinc-dependent metalloprotease domain, and a prodomain possibly involved in maintaining protease latency. However, ovastacin was found to have an additional 150 amino acid C-terminal domain not found in other astacins. Quesada et al. also showed that the protein has metalloprotease activity, and that it was expressed in no normal tissues other than ovary. They suggested that its normal function of ovastacin might be similar to the astacin family “hatching enzymes” of lower species. Additionally, they found that it was expressed in some cancer cells, including lymphoma and leukemia cells lines, but only two of five ovarian carcinomas tested, and that was only detectable using RT-PCR. Other groups have more recently referred to ovastacin as “Astacin Like protein” (ASTL).

Ovastacin, also referred to as ZEP, SAS1R, and SAS1B herein, has recently been to be involved in preventing polyspermy by acting as an endoprotease to cleave the zona pellucida glycoprotein ZP2 at the time of gamete fusion (Burkart et al., 2012, J. Cell Biol., 197:37-44).

At about the same time Quesada discovered “ovastacin”, another group isolated it and referred to it at first as Zinc Endopeptidase (ZEP) (Mandal et al., published on Aug. 31, 2006, PCT Pat. Pub. No. WO 2006/091535) and later as Sperm Acrosomal SLLP1 Receptor (“SAS1R”), because it was an oocyte protein that they found interacted with the sperm protein SLLP1 (Mandal et al., 2008, Biol. of Reproduction, 78:69:72; Herr et al., PCT Pat. Pub. No. WO 2010/054187, published May 14, 2010).

Mandal et al., (PCT Pat. Pub. No. WO 2006/091535) showed that ZEP had 2 variants, a sequence indicating a predicted transmembrane domain, a cleavage site, and a zinc binding signature. They pointed out that it was homologous to the hatching enzyme EHE7 of the Japanese eel Anguilla japonica and hypothesized that it may be performing a similar function in mouse embryo development. The bioinformatic analysis of Mandal showed that the protein has two glycosylation sites, phosphorylation sites, and myristylation sites. They noted that the sites were suggestive of a membrane protein and that transmembrane topology also predicted a strong transmembrane domain at the N-terminal of the protein. Their data showed that it was egg specific, and that it localized on the egg surface in the microvillar region, but was developmentally regulated. The data also suggested interactions between ZEP and the sperm surface protein SLLP1.

In Mandal et al. (Biology of Reproduction, 78:69-72, 2008), the group began referring to ZEP as SAS1R. They found that SAS1R localized on the microvillar domain of mature live oocytes and was significantly lost after fertilization, being virtually undetectable in blastocysts. They showed that transfection of CHO-K1 cells with a full length SAS1R cDNA construct allowed the protein to be expressed on the surface of non-permeabilized cells, indicating the presence of an active transmembrane domain. They also described protease characteristics and the ability of SAS1R to act as the receptor for the sperm protein SLLP1.

Herr et al. (PCT Pat. Pub. No. WO 2010/054187; published May 14, 2010) found that native SAS1R showed binding to recombinant SLLP1 and that bound recombinant SAS1R captured recombinant SLLP1; SAS1R and SLLP1 revealed molecular binding properties by yeast two hybrid analysis; immunoprecipitation of recombinant SAS1R recovered recombinant SLLP1 and immunoprecipitated recombinant SLLP1 recovered recombinant SAS1R from rabbit reticulocyte extract; recombinant SLLP1 binds to oocyte microvillar domain and co-localizes with native SAS1R; recombinant SAS1R binds to acrosome of sperm and co-localizes with native SLLP1; native SLLP1 from sperm acrosomal matrix localizes with native SAS1R; and native SAS1R and native SLLP1 are co-precipitated from mixtures of non-ionic detergent extracts of oocytes and sperm. Herr et al. also showed that SAS1R is localized on live human eggs retrieved for in vitro fertilization and that administration of exogenous SAS1R to a subject elicits an immune response against SAS1R. They also demonstrated that SAS1R protein first arises in bilaminar secondary follicles during postnatal oogenesis, in pubertal oogenesis, as well as adult oogenesis. The pattern is uniform irrespective of the age of the animal. In adult mouse ovaries, SAS1R staining is restricted to oocytes within secondary follicles and all subsequent stages. Primordial oocytes and primary oocytes do not stain for SAS1R at any developmental stage. They found that the only cell type in the ovary that stained for SAS1R was oocytes and that the presence of SAS1R was developmentally regulated.

Herr et al. (International Publication Number WO 2012/019184, published Feb. 9, 2012) showed that SAS1R is expressed in various cancers, including ovarian, uterine, lung and bladder cancer cells and tumors, as well as in an ovarian cancer stem cell line, but not its differentiated counterpart. They also showed it is a cell surface protein.

Ribosome-inactivating proteins (RIPs) are plant enzymes that damage ribosomes, and possibly other substrates, in an irreversible manner. RIPs are mainly divided into type 1, which consist of a single-chain protein (e.g., Saporin-S6 (SAP; from seeds of Saponaria officinalis L.)), and type 2, which are composed of an enzymatic A-chain linked to a B-chain possessing lectin properties. The interest in RIPs derives mostly from potential applications after being linked to appropriate carriers, such as monoclonal antibodies (mAbs) or other molecules, to obtain conjugates that are specifically toxic to target cells.

Given the lack of definitive diagnostic tests for cancer and the poor prognosis for patients with metastatic disease, there is a long felt need in the art for diagnostic tests for cancers.

There is also a long felt need in the art to identify and use cancer biomarkers and to find methods to regulate these biomarkers, including targeting the biomarker for treatment and prevention of cancer.

### SUMMARY OF THE INVENTION

The present invention provides compositions and methods useful for treating cancer, based on the unexpected results disclosed herein that the oocyte protein, SAS1B, also referred to as ovastacin, ZEP, and SAS1R, aberrantly expressed on the surface of cancer cells can be targeted by immunotoxins to kill the cell or to inhibit its proliferation. Herr et al. (International Publication Number WO 2012/019184, published Feb. 9, 2012), the entirety of which is incorporated by reference herein, showed that SAS1R is expressed in multiple types of cancers and is expressed at the cell surface, thus making SAS1R the first cancer-oocyte antigen/biomarker discovered. Cancer-oocyte antigens/biomarkers are defined as proteins expressed among normal tissues only in the oocyte and only at specific stages of folliculogenesis. The expression of a cell surface protein, normally restricted to the oocyte, in various tumors offers striking opportunities for a tumor selective therapy when accompanied by a companion diagnostic to identify subjects expressing the cancer-oocyte antigen target. Therefore, SAS1B is a useful biomarker for detecting and diagnosing cancer.

On embodiment provides an immunotoxin molecule comprising an antibody, or a binding fragment thereof, specific for SAS1B antigen conjugated to a cytotoxic agent. In one embodiment, the cytotoxic agent is a Type I ribosome inactivating protein. In another embodiment, the Type I ribosome inactivating protein is saporin. Another embodiment provides the use of trichosanthin and luffin as the cytotoxic agent. In one embodiment, the is monoclonal, polyclonal, chimeric, human, or humanized. In another embodiment, the antibody binding fragment is F(ab′)₂, F(ab)₂, Fab′, or Fab.

One embodiment provides a composition comprising the immunotoxin and a physiologically acceptable carrier.

Another embodiment provides a method of killing, inhibiting or sloughing off of cancer cells comprising administering to a subject in need thereof at least one immunotoxin molecule described herein. In one embodiment, the cancer is selected from the group consisting of carcinoma (e.g., head and neck squamous cell carcinoma), sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, neuroblastoma, melanoma, breast cancer, prostate cancer, pancreatic cancer, kidney cancer, bladder cancer and endometrioid carcinoma.

Another embodiment provides a method to treat cancer, comprising administering a therapeutically effective dose of an immunotoxin conjugate to a subject in need thereof, wherein said immunotoxin conjugate comprises an antibody, or a binding fragment thereof, conjugated to a cytotoxic agent, wherein said antibody of said immunotoxin conjugate binds to SAS1B on a cancer cell, and wherein the binding of said immunotoxin molecule to the SAS1B molecule on a cell results in the killing, sloughing off or inhibiting of proliferation of the SAS1B expressing cell. In one embodiment, the cytotoxic agent is a Type I ribosome inactivating protein. In another embodiment, the Type I ribosome inactivating protein is saporin. In one embodiment, the antibody is monoclonal, polyclonal, chimeric, human, or humanized. In other embodiment, the antibody binding fragment is F(ab′)₂, F(ab)₂, Fab′, or Fab. In one embodiment, the cancer is selected from the group consisting of carcinoma (e.g, head and neck squamous cell carcinoma), sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, neuroblastoma, melanoma, breast cancer, prostate cancer, pancreatic cancer, kidney cancer, bladder and endometrioid carcinoma.

One embodiment provides a method treat cancer, comprising administering a therapeutically effective dose of a first antibody, or fragment thereof, which binds to SAS1B on a cancer cell, and a second antibody that binds to the first antibody, wherein the second antibody is conjugated to a cytotoxic agent. In one embodiment, one or both antibodies are is monoclonal, polyclonal, chimeric, human, or humanized. In another embodiment, the antibody binding fragment is F(ab′)₂, F(ab)₂, Fab′, or Fab. In one embodiment, the cytotoxic agent is a Type I ribosome inactivating protein. In another embodiment, the Type I ribosome inactivating protein is saporin.

One embodiment provides a method to diagnose ovarian, uterine, bladder, kidney cancer, pancreatic cancer or head and neck squamous cell carcinoma (HNSCC) comprising measuring SAS1B in a biological sample from kidney, pancreas or head and neck of a subject, and diagnosing kidney cancer, pancreatic cancer or head and neck squamous cell carcinoma (HNSCC) in said subject based on the presence of SAS1B in the biological sample from kidney, pancreas or head and neck of the subject. In one embodiment, the subject is mammalian. In another embodiment, the SAS1B is SAS1B protein, miRNA or mRNA. In one embodiment, the detection comprises analyzing the results with an analytical device and program. In one embodiment, the analytical device comprises a computer. In one embodiment, the analytical device comprise a sequence analyzer. In one embodiment, the level of SAS1B protein, miRNA or mRNA is quantified with an analytical device and program. In another embodiment, the SAS1B protein, miRNA, or mRNA is detected using a method selected from the group consisting of ELISA, immunoassay, immunofluorescence, immunohistochemistry, immunoprecipitation, northern blot, western blot, PCR, mass spectrometry and surface Plasmon resonance. In one embodiment, the sample is tissue biopsy.

In one embodiment, contacting a SAS1B positive cancer cell with an antibody directed against SAS1R causes a change in shape of the cell. In one embodiment, contacting a SAS1B positive cancer cell with an antibody directed against SAS1R causes a change in size of the cell. In one aspect, a treated cell decreases in size.

In one aspect, an antibody directed against SAS1B can direct endocytosis of the complex in a cell wherein said cell has surface SAS1B. In one aspect, an antibody directed against SAS1B is conjugated to a drug or toxin. In one aspect, a secondary antibody which binds to the primary antibody to SAS1B is used, wherein a drug or toxin is conjugated to the secondary antibody. One or more these antibody/conjugate or antibody/secondary antibody/conjugate complexes can be used. The complex can include a drug or toxin conjugated to the antibody directed against SAS1B or to a secondary antibody that will bind to the antibody directed against SAS1B. In one aspect, the conjugated drug or toxin is saporin. In one aspect, the directed endocytosis results in killing the cell. In one aspect, the saporin-conjugated antibody mediates sloughing of SAS1B positive cells. In one aspect, the cell is a cancer cell. One of ordinary skill in the art will appreciate that different kinds of antibodies can be used, including monoclonal antibodies and polyclonal antibodies. In one aspect, the compounds and compositions of the invention are useful for inducing growth arrest of SAS1B positive cells. Incorporation of the antibody and drug/toxin conjugate into the cell is useful for killing the targeted cell. Cells not expressing SAS1B are not targets.

In one aspect the antibody against SAS1B contains complement binding capacity and binds to tumor cell surface SAS1B whereupon endogenous and natural components of the complement cascade induce tumor cell killing.

The present invention further provides compositions and methods useful for precision, personalized medicine. In one embodiment, the present invention provides compositions and methods useful for selecting a subject with cancer who will be responsive to treatment with an antagonist or inhibitor of SAS1B, comprising detecting the presence of SAS1B protein, miRNA or mRNA in a sample from the subject, wherein the presence of SAS1B protein, miRNA or mRNA in the sample indicates that the subject will be responsive to treatment with an antagonist or inhibitor of SAS1B. The sample specimen may be from among the tissue fluids of blood, serum, saliva, semen, urine or extracts of tumor biopsy.

The present invention also provides compositions and methods useful for preventing and for treating SAS1B positive cancer.

In one embodiment, the antibody is selected from the group consisting of a single chain antibody, a monoclonal antibody, a bi-specific antibody, a chimeric antibody, a synthetic antibody, a polyclonal antibody, or a humanized antibody, or active fragments or homologs thereof. In one aspect, the antibody binds to one or more SAS1B protein fragments selected from the group consisting of amino acids 1-25, 26-50, 51-75, 76-100, 101-125, 126-150, 151-175, 176-200, 201-225, 226-250, 251-275, 276-300, 301-325, 326-350, 351-375, 376-400, 401-425, and 426-431 of SAS1B human variant 1 (SEQ ID NO:23). In one aspect, the antibody binds to one or more SAS1B protein fragments selected from the group consisting of amino acids 1-20, 21-40, 41-60, 61-80, 81-100, 101-120, 121-140, 141-160, 161-180, 181-200, 201-220, 221-240, 241-260, 261-280, 281-300, 301-320, 321-340, 341-360, 361-380, 381-400, 401-420, 421-431, and 411-431 of SAS1B human variant 1 (SEQ ID NO:23).

The present invention further provides compositions and methods for killing cancer cells and for inhibiting proliferation of cancer cells. The method for inhibiting proliferation or killing a SAS1B positive cancer cell comprises contacting said cancer cell with an effective amount of antibody directed against SAS1B or a fragment thereof, wherein the antibody directed against SAS1B or a fragment thereof binds with SAS1B, thereby inhibiting proliferation or killing a cancer cell. In one embodiment, the killing is complement dependent cytotoxicity. In one aspect, complement is supplemented. In one embodiment, the invention provides compositions and methods for lysing cancer cells with polyclonal or monoclonal antibodies directed against SAS1B, or fragments thereof, in the presence of complement. In one embodiment, the cancer cell being killed or inhibited from proliferating is selected from the group consisting of carcinoma, sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, kidney cancer, bladder and endometrioid carcinoma. In one aspect, the antibody is conjugated to another molecule or structure. In one aspect, the other molecule or structure is selected from the group consisting of an antibody, a protein, a prodrug, a drug, a toxin, a protein toxin, a liposome, a radioactive isotope, and an enzyme.

In one embodiment, the antibody directed against SAS1B is conjugated to a toxin or drug directly, or indirectly when the drug or toxin is conjugated to a secondary antibody which binds to the antibody directed against SAS1B. In one aspect, a secondary antibody conjugated with a drug or toxin is used wherein said secondary antibody targets the antibody directed against SAS1B. In one aspect, a subject in need thereof is treated by administering a pharmaceutical composition comprising at least one antibody directed against SAS1B, optionally conjugated to a drug or toxin. In one aspect, a secondary antibody that binds to the antibody directed against SAS1B is also used, and optionally the secondary antibody is conjugated to a drug or toxin.

The present invention provides compositions and methods useful for treating SAS1B positive cancers using an immunotoxin of the invention. One of ordinary skill in the art will appreciate that other drugs and toxins than just those described herein can be used as well, as can additional therapeutic agents. Those agents can be added to the pharmaceutical composition of the invention comprising at least one antibody directed against SAS1B, which is optionally conjugated to a toxin or drug, and optionally a secondary antibody is used which is also optionally conjugated to a toxin or drug. The two antibodies (a first and a second) can be administered simultaneously or separately.

The present application further discloses the unexpected result that SAS1B is expressed in kidney and pancreatic cancers and head and neck squamous cell carcinoma. The present invention therefore encompasses compositions and methods for diagnosing and treating kidney cancer and pancreatic cancers and head and neck squamous cell carcinoma.

The present invention further provides compositions and methods for testing tumor cells in vitro for sensitivity to treatment by the immunotoxins of the invention before a subject with the tumor is treated. The assays provided herein demonstrate the ranges of antibodies and conjugates that are used and can be effective. Disclosed herein is the surprising result of low dosages that are effective.

Various aspects and embodiments of the invention are described in further detail below.

### BRIEF DESCRIPTION OF THE DRAWINGS

FIG. 1 shows MMMT 538 cells that were incubated with antibody to SAS1B at 4° C., warmed to 37° C. for 15 min, and then stained for the location of the SAS1B antibody. Small SAS1B antibody-positive vesicles were observed at the cell periphery just beneath the cell membrane

FIG. 2 shows an MMMT 539 cell after similar treatment, but after 60 minutes of incubation at 37° C. SAS1R antibody positive vesicles were found to be larger and more deeply internalized in the cytoplasm. These results indicate that SAS1B is internalized in MMMT cancer cells following ligand binding.

FIG. 3 demonstrates that SAS1B positive vesicles co-localized with components of the early and late arms of the endocytic pathway. For example, yellow vesicles (arrowheads) represent where green SAS1R positive vesicles co-localized with red vesicles stained for EEA1, a marker of the early arm of the endocytic pathway. Similar results have been noted with LAMP1, a marker of the late arm of the endocytic pathway. Together these results indicate cell surface SAS1B is internalized and fuses with compartments of the early and late arms of the endocytic system.

FIG. 4 shows the 310 bp c-term SAS1B amplimer in MMMT 539 cells, but not in the virus transformed uterine stromal cell line MAD10, demonstrating that MMMT 539 cells express SAS1B, whereas MAD10 cells do not.

FIG. 5 demonstrates, through an indirect saporin assay employing the sulforhodamine B screening method to quantify cell density and growth arrest performed with MMMT 539 and MAD 10 cells, that the SAS1B pathway can be used for intracellular delivery of a cytotoxic cargo. Further, the immune sera to SAS1B [IM-Zap] at 0.01 uM concentration provides maximum cell killing effect when the complex with a secondary antibody conjugated to saporin (held constant at approx. 5 nm). The saporin immunotoxin mediated sloughing of M539 uterine cancer cell lines.

FIG. 6 demonstrates the specificity of the reaction to cell surface SAS1B by showing that there is no effect of any concentrations of IM-Zap on cell growth using a cell line that does not express SAS1B. No cell killing and growth arrest was demonstrated with control endometrial stromal MAD10 cells in cells treated with the anti-SAS1B and saporin immunotoxin.

FIGS. 7 and 8 demonstrate that IM sera to SAS1B at 0.01 uM concentration shows maximum cell killing effects when the saporin immunotoxin is at a nanomolar concentration.

FIG. 9. Oncomine (a cancer microarray database and web-based data-mining platform aimed at facilitating discovery from genome-wide expression analyses) interrogations demonstrate that SAS1B (ASTL) is elevated in clear cell renal cell carcinomas.

FIG. 10. Demonstration of SAS1B in renal cancer specimens. cDNA from clear cell renal tumors from patients was used in the PCR assay using c-term SAS1B primers (panel A) and, to ensure integrity of RNA, GAPDH primers were used in panel B. M539 uterine cancer cell lines known to express SAS1B were used as a positive control as seen in lane 9. Normal kidney cDNA served as a negative tissue control in 11 and does not express SAS1B.

FIG. 11 demonstrates that SAS1B mRNA and SAS1B proteins are observed in 100% of HNSCC cells lines investigated.

FIG. 12 demonstrates that 100% of HNSCC human tumor specimens investigated were positive for SAS1B.

FIG. 13 demonstrates that SAS1B transcripts were identified in pancreatic cancer cell lines.

FIG. 14 demonstrates that SAS1B was identified in pancreatic tumor specimens.

FIG. 15 provides pancreatic PCR controls.

### DETAILED DESCRIPTION

### Summary of SEQ ID NOs. and Description Thereof

SEQ ID NO:1—mouse (“m”) MET normal nucleic acid sequence
SEQ ID NO:2—mouse MET normal amino acid sequence
SEQ ID NO:3—mouse MET variant nucleic acid sequence
SEQ ID NO:4—mouse MET variant amino acid sequence
SEQ ID NO:5—mouse SAS1R Variant 2 Normal nucleic acid sequence (formerly called ZEP-Normal)
SEQ ID NO:6—mouse SAS1R Variant 2 Normal amino acid sequence (formerly called ZEP-Normal)
SEQ ID NO:7—mouse SAS1R Variant 5 nucleic acid sequence (formerly called ZEP Variant 1)
SEQ ID NO:8—mouse SAS1R Variant 5 amino acid sequence (formerly called ZEP Variant 1)
SEQ ID NO:9—mouse SAS1R Variant 3 nucleic acid sequence (formerly called ZEP Variant 2)
SEQ ID NO:10—mouse SAS1R Variant 3 amino acid sequence (formerly called ZEP Variant 2)
SEQ ID NO:11—mouse SLLP1 nucleic acid sequence
SEQ ID NO:12—mouse SLLP1 amino acid sequence
SEQ ID NO:13—human (“h”) SLLP1 nucleic acid sequence
SEQ ID NO:14—human SLLP1 amino acid sequence
SEQ ID NO:15—mouse SLLP2 nucleic acid sequence
SEQ ID NO:16—mouse SLLP2 mature protein amino acid sequence
SEQ ID NO:17—human SLLP2 nucleic acid sequence
SEQ ID NO:18—human SLLP2 amino acid sequence
SEQ ID NO:19—mouse SAS1R Variant 1 amino acid sequence
SEQ ID NO:20—mouse SAS1R Variant 4 amino acid sequence
SEQ ID NO:21—mouse SAS1R Variant 6 amino acid sequence
SEQ ID NO:22—human SAS1R nucleic acid sequence (GenBank accession no. NM_{—}001002036, 1296 by mRNA)
SEQ ID NO:23—human SAS1R amino acid sequence (GenBank accession no. NP_{—}001002036.3, 431 amino acids)
SEQ ID NO:24—HELMHVLGFWH (motif in SAS1R with histidine residues for Zn coordination and conserved catalytic residue, E (glutamic acid), forms part of the catalytic pocket along with a tyrosine zinc ligand embedded in the motif SVMHY (SEQ ID NO:25).
SEQ ID NO:25—SVMHY (motif in SAS1R associated with the catalytic pocket)
SEQ ID NO:26—HEXXHXXGXXH (the consensus motif of SEQ ID NO:24 can have residues which can be substituted with any amino acid, as indicated by “X”, that does not ablate the function of that motif).
SEQ ID NO:27—SXMHY (the consensus motif of SEQ ID NO:25 can have residues which can be substituted with any amino acid, as indicated by “X”, that does not ablate the function of that motif).

1F primer-
SEQ ID NO: 28
GCGCCCCTGGCCTCCAGCTGCGCA
2R primer-
SEQ ID NO: 29
CACGACACCACTACCACCCATGGG
3F primer-
SEQ ID NO: 30
GGCTGCAGCCCAAGTGGCCCCAGG
4R primer-
SEQ ID NO: 31
AGCAACACCGGGGGCACCTGCTCC
5F primer-
SEQ ID NO: 32
GAGGTCCCCTTCCTGCTCTCCAGC
6R primer-
SEQ ID NO: 33
GGCATGGGACCCTCTCCCACGGGG.

SEQ ID NOs:1-18 are the same sequences as SEQ ID NOs:1-18 of international patent application WO 2006/091535 (PCT/US2006/005970; Mandal et al.; published Aug. 31, 2006), in which SAS1R was referred to as ZEP. WO 2006/091535 is incorporated by reference in its entirety herein.

SEQ ID NOs: 1-27 are the same 27 sequences used in international patent application PCT/US/2009/063540 (Herr et al.), filed Nov. 6, 2009, published on May 14, 2010 as WO 2010/054187. A U.S. application (Ser. No. 12/613,947) claiming priority to the PCT application published on Jul. 22, 2010 as Pub. No. US 2010/0183617.

SEQ ID NOs: 28-33 are novel primers for detecting SAS1R.

SEQ ID NOs:1-33 are all used in Herr et al. (International Publication Number WO 2012/019184, published Feb. 9, 2012).

### ABBREVIATIONS AND ACRONYMS

a. a.—amino acid(s)
ADEPT—antibody-directed enzyme prodrug therapy
ASTL—astacin-like protein; this name is now commonly used and accepted and refers to the same protein also referred to as ovastacin, ZEP, SAS1R and SAS1B
BSA—bovine serum albumin
CDC—complement-dependent cytotoxicity
Co-IP—co-immunoprecipitation
FITC—fluorescein isothiocyanate
FRET—fluorescence resonance energy transfer
FW—Far Western
GV—germinal vesicle
h—human (also hour)
HEK—human embryonic kidney
HPLC—reversed-phase high-pressure liquid chromatography
HS—high stringency
I—induced or immune
IF—Indirect immunofluorescence
IP—immunoprecipitation
IPTG—Isopropyl-β-D-thiogalactopyranoside
LB—Luria broth
LC/MS means liquid chromatography/mass spectrometry
LNA—locked nucleic acids
LS—low stringency
IM—immune
m—mouse
MET—mouse egg-specific TolA (referred to in the provisional application as a Colcin-like uptake protein or Colicin uptake protein)
min—minute
miRNA—microRNA
MMMT—malignant mixed mullerian tumor
NGS—normal goat serum
OL—overlay
P—purified
PI—pre-immune
PBS—phosphate-buffered saline
PBST—phosphate buffered saline with 0.05% Tween 20
PVA—polyvinylalcohol
rec—recombinant (rec is used interchangeably with “r”)
SAS1B—see “SAS1R”
SAS1R—Sperm Acrosomal SLLP1 Receptor; previously referred to as ZEP and originally discovered and called “Ovastacin” by Quesada et al.; also referred to as SAS1B; the gene encoding SAS1R is referred to as ASTL
rSAS1R—recombinant SAS1R
sec—second(s)
SLLP—sperm lysozyme-like protein
SPECT—single photon emission computed tomography
SPR—surface plasmon resonance
U—uninduced
ZEP—zinc endopeptidase (referred to in the provisional application as zinc peptidase, or ZP; used interchangeably with SAS1R, SAS1B, ovastacin and ASTL)
ZFE—zona free egg
ZIE—zona intact egg

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the invention or a prodrug of a compound of the invention to a subject in need of treatment.

As used herein, the term “aerosol” refers to suspension in the air. In particular, aerosol refers to the particlization or atomization of a formulation of the invention and its suspension in the air.

As used herein, an “agonist” is a composition of matter which, when administered to a mammal such as a human, enhances or extends a biological activity attributable to the level or presence of a target compound or molecule of interest in the mammal.

An “antagonist” is a composition of matter which when administered to a mammal such as a human, inhibits a biological activity attributable to the level or presence of a compound or molecule of interest in the mammal.

As used herein, “alleviating a disease or disorder symptom,” means reducing the severity of the symptom or the frequency with which such a symptom is experienced by a patient, or both.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

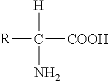

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fᵥ, single chain Fᵥ, complementarity determining regions (CDRs), V_{L} (light chain variable region), V_{H} (heavy chain variable region), Fab, F(ab′)₂ and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)₂ a dimer of Fab which itself is a light chain joined to V_{H}—C_{H1} by a disulfide bond. The F(ab′)₂ may be reduced under mild conditions to break the disulfide linkage in the hinge region, thereby converting the F(ab′)₂ dimer into an Fab₁ monomer. The Fab₁ monomer is essentially an Fab with part of the hinge region (see, FUNDAMENTAL IMMUNOLOGY, 3RD ED., W. E. Paul, ed, Raven Press, N.Y. (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies.

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Bire, et al., Science 242:423 (1988) and Huston, et al., Proc. Nat'l Acad. Sci. USA 85:5879 (1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin.

Humanized antibodies have been referred to as “reshaped” antibodies. Manipulation of the complementarity-determining regions (CDR) is a way of achieving humanized antibodies. See, for example, Jones, et al., Nature 321:522 (1988) and Riechmann, et al., Nature 332:323 (1988), both of which are incorporated by reference herein. For a review article concerning humanized antibodies, see Winter & Milstein, Nature 349:293 (1991), incorporated by reference herein.

The term “joined” in the context of the immunotoxins of this invention encompasses the linking of moieties (typically an antibody and a toxin) through covalent bonding, including disulfide bonding; hydrogen bonding; electrostatic bonding; recombinant fusion; and conformational bonding, e.g., antibody-antigen, and biotin-avidin associations.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, skin, hair, tissue, blood, plasma, cells, sweat and urine.

“C19” and “C23” are names which are also used for “SLLP1” and SLLP2″.

The term “cancer”, as used herein, is defined as proliferation of cells whose unique trait—loss of normal controls—results in unregulated growth, lack of differentiation, local tissue invasion, and metastasis. Examples include but are not limited to, melanoma, breast cancer, prostate cancer, ovarian cancer, uterine cancer, cervical cancer, skin cancer, pancreatic cancer, colorectal cancer, renal cancer and lung cancer.

As used herein, the term “carrier molecule” refers to any molecule that is chemically conjugated to the antigen of interest that enables an immune response resulting in antibodies specific to the native antigen.

The term “cell surface protein” means a protein found where at least part of the protein is exposed at the outer aspect of the cell membrane. Examples include growth factor receptors.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule. Chemical conjugation also includes non-covalent linkage between the antigen and the carrier molecule.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:
I. Small aliphatic, nonpolar or slightly polar residues:
   Ala, Ser, Thr, Pro, Gly;
II. Polar, negatively charged residues and their amides:
   Asp, Asn, Glu, Gln;
III. Polar, positively charged residues:
   His, Arg, Lys;
IV. Large, aliphatic, nonpolar residues:
   Met Leu, Ile, Val, Cys
V. Large, aromatic residues:
   Phe, Tyr, Trp

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting aberrant SAS1R expression due to cancers expressing SAS1R. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm which is capable of regulating a biochemical pathway.

As used herein, the phrases “egg protein” or “egg-specific protein” refer to proteins which are expressed exclusively or predominately in eggs or ovaries. The proteins need not be expressed at all stages of egg or ovarian development.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

By the term “immunizing a subject against an antigen” is meant administering to the subject a composition, a protein complex, a DNA encoding a protein complex, an antibody or a DNA encoding an antibody, which elicits an immune response in the subject, and, for example, provides protection to the subject against a disease caused by the antigen or which prevents the function of the antigen.

The term “immunologically active fragments thereof” will generally be understood in the art to refer to a fragment of a polypeptide antigen comprising at least an epitope, which means that the fragment at least comprises 4 contiguous amino acids from the sequence of the polypeptide antigen.

As used herein, the term “induction of apoptosis” means a process by which a cell is affected in such a way that it begins the process of programmed cell death, which is characterized by the fragmentation of the cell into membrane-bound particles that are subsequently eliminated by the process of phagocytosis.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

The term “inhibit a complex,” as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

By ‘interaction” between a sperm protein and an egg protein is meant the interaction such as binding which is necessary for an event or process to occur, such as sperm-egg binding, fusion, or fertilization. In one aspect, the “interaction” may be similar to receptor-ligand type of binding or interaction.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nasal administration” in all its grammatical forms refers to administration of at least one compound of the invention through the nasal mucous membrane to the bloodstream for systemic delivery of at least one compound of the invention. The advantages of nasal administration for delivery are that it does not require injection using a syringe and needle, it avoids necrosis that can accompany intramuscular administration of drugs, and trans-mucosal administration of a drug is highly amenable to self administration.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

An “oocyte” as used herein can be categorized more specifically several ways. A “naked oocyte” is defined as a female germ cell that is not surrounded by a continuous sheet of nurse granulose cells. A “primordial oocyte” is defined as a female germ cell that is surrounded by a single layer of squamous nurse granulosa cells. A “primary oocyte” is defined as a female germ cell that is surrounded by a single layer of cuboidal nurse granulose cells. A “secondary oocyte” is defined as a female germ cell that is surrounded by two layers of cuboidal granulose cells. A “preeantral oocyte” is defined as a female germ cell that is surrounded by three or more layers of granulose cells but without an antral space. An “antral oocyte” is defined as a female germ cell that is surrounded by three or more layers of granulosa cells and contains evidence of antral fluid spaces.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

“Prozone” or “prozone effect”, also referred to as the “Hook effect”, is where in an agglutination or precipitation reaction, prozone or prezone is the zone of relatively high antibody concentrations within which no reaction occurs. For example, in an agglutination test, a person's serum (which contains antibodies) is added to a test tube which contains a particular antigen. There are many types of different antibodies present in a person's serum, and there might be one particular antibody that will bind to antigen present in the tube. There are also other types of antibodies which would not bind the antigen. The antibody-antigen complex forms an agglutinate. In some cases, the concentration of antibody specific for a particular antigen is too low compared to other types of antibodies, and when this occurs, the small portion of agglutination that took place will be masked by larger aggregate of non-binding antibodies, and the mixture remain as fluid. The agglutination which took place is invisible, and this is called prozone. This can lead to false negative result. Therefore in each agglutination test, dilution of the antibody-antigen mixture is done to a certain level, until agglutination can be seen, or otherwise the test is negative. Prozone effects are also seen in indirect immunotoxin experiments, where excess of free primary antibody results 1) in occupation and saturation of cell surface receptors by antibodies that remain unbound to secondary immunotoxins and 2) by formation in solution of primary antibody-immunotoxin complexes that do not reach the cell surface to bind with receptor (SAS1B). When prozone effects are noted, the biological effects (growth arrest) of target directed antibodies typically are observed as equivalent stoichiometries of primary antibody and secondary immunotoxin are approached.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-β-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “SAS1B positive cancer” as used herein refers to a cancer wherein cells of the cancer express or comprise SAS1B. The terms “SAS1B positive cancer” and “SAS1R positive cancer” are used interchangeably herein. The terms should be used in the context as presented and can include cells with SAS1B on the cell surface and in some cases may refer to cells expressing SAS1B mRNA or protein, or peptides with varied sequences due to alternative splicing.

“SLLP1” and SLLP2″ are also referred to as “C19” and “C23”, respectively.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal Such animals include mammals, preferably a human.

As used herein, a “subject in need thereof” is a patient, animal, mammal, or human, who will benefit from the method of this invention.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO₄, 1 mM EDTA at 50° C. with washing in 2× standard saline citrate (SSC), 0.1% SDS at 50° C.; preferably in 7% (SDS), 0.5 M NaPO₄, 1 mM EDTA at 50° C. with washing in 1×SSC, 0.1% SDS at 50° C.; preferably 7% SDS, 0.5 M NaPO₄, 1 mM EDTA at 50° C. with washing in 0.5×SSC, 0.1% SDS at 50° C.; and more preferably in 7% SDS, 0.5 M NaPO₄, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

By the term “vaccine,” as used herein, is meant a composition which when inoculated into a subject has the effect of stimulating an immune response in the subject, which serves to fully or partially protect the subject against a condition, disease or its symptoms. In one aspect, the condition is conception. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Embodiments

The present invention relates to immunotoxins that effectively kill malignant cells having a given marker. The immunotoxins are reagents that comprise antibodies conjugated to a cytotoxic agent or fragments thereof. The antibodies are capable of binding with a chosen tumor cell, and thereby confer little non-specific toxicity of the immunotoxin in a host.

A. Antibodies

Antibodies refer to polypeptides substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

A variety of methods for producing polyclonal and monoclonal antibodies are known in the art. See, e.g., Goding, MONOCLONAL ANTIBODIES; PRINCIPLES AND PRACTICE, Academic Press, 2nd Edition (1986); and Harlow & Lane. A monoclonal antibody directed against or reactive with, for example, human cells expressing a desired antigen is obtained by using combinations of immunogens to immunize mice and screening hybridoma supernatant against cells which express the desired antigen or by a screening assay designed to be specific for monoclonal antibodies directed against the antigen of interest. Useful cell lines for screening for the antibodies of this invention are readily available or obtained. Such cells include the Burkitt's lymphoma cell lines Daudi, and Raji.

Recombinant DNA methodologies can be used to synthesize antibodies of this invention. For example, an antibody portion of an immunotoxin for use in humans is a “humanized” antibody against a cell antigen which contains murine complementarity-determining regions (CDRs) combined with human variable region frameworks and human constant regions.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369). Detailed methods for preparation of chimeric (humanized) antibodies can be found in U.S. Pat. No. 5,482,856.

In another embodiment, this invention provides for fully human antibodies. Human antibodies consist entirely of characteristically human polypeptide sequences. The human antibodies of this invention can be produced in using a wide variety of methods (see, e.g., Larrick et al, U.S. Pat. No. 5,001,065, for review).

The antibody moieties of this invention can be single chain antibodies.

Antibodies directed against proteins, polypeptides, or peptide fragments thereof of the invention may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

In one embodiment, antibodies, or antisera, directed against SAS1R or a homolog or fragment thereof, are useful for blocking the activity of SAS1R, including its ability to interact with other molecules or cells.

Fragments of SAS1R may be generated and antibodies prepared against the fragments. Assays are provided herein to determine whether an antibody directed against SAS1R, or a fragment thereof, have the ability to detect SAS1R, to inhibit SAS1R activity, or regulate SAS1R function.

The assays include measuring the ability of SAS1R to bind with or interact with SLLP proteins, as well as the ability of an antibody to block SAS1R's role in fertilization. For example, in vitro fertilization assays are described herein using an antibody directed SAS1R and this type of assay can be used to test the ability of new antibodies to block SAS1R's function. These same assays can be used to test any compound or agent's ability to disrupt SAS1R's interaction with a SLLP protein or to inhibit fertilization. Protease assays for measuring SAS1R protease activity are also available when needed to confirm that a fragment or homolog of SAS1R maintains the same activity as the parent SAS1R molecule.

Various methods of preparing fragments of SAS1R and making antibodies against SAS1R are available and these methods can be used to map the various regions of SAS1R that are susceptible to inhibition by an antibody.

For example, fragments of SAS1R can be prepared for use as an antigen, such as wherein the antibody binds to one of more fragments comprising amino acids 1-25, 26-50, 51-75, 76-100, 101-125, 126-150, 151-175, 176-200, 201-225, 226-250, 251-275, 276-300, 301-325, 326-350, 351-375, 376-400, and 401-414 of SAS1R Variant 2 (SEQ ID NO:6) or wherein the antibody binds to one or more fragments comprising amino acids 1-25, 26-50, 51-75, 76-100, 101-125, 126-150, 151-175, 176-200, 201-225, 226-250, 251-275, 276-300, 301-325, 326-350, 351-375, 376-400, 401-425, and 426-435 of SAS1R Variant 1 (SEQ ID NO:19) or wherein the antibody binds to amino acids 1-25, 26-50, 51-75, 76-100, 101-125, 126-150, 151-175, 176-200, 201-225, 226-250, 251-275, 276-300, 301-325, 326-350, 351-375, 376-400, 401-425, and 426-431 of SAS1R Variant 1 (SEQ ID NO:23). The invention further encompasses fragments comprising 20 amino acids, such as amino acid residues 1-20, 21-40, 41-60, etc. Such techniques can also be applied to the full-length protein.

Of course, these fragments can also be prepared to yield overlapping sequences and longer and shorter fragments can be prepared. For example, as described herein, interaction experiments between SLLP1 and SAS1R indicate there are at least two binding regions between the two proteins when they interact, which may have different functions. There, fragments encompassing sections of the more N-terminal region of SAS1R or the more C-terminal region of SAS1R can be prepared, such as wherein the antibody binds to amino acids about 1 to about 121 (N-terminal) or an antibody which binds to about 204 to about 414 (more C-terminal) of SAS1R (SEQ ID NO:6) or an antibody which binds to similar regions of SEQ ID NO:23 (human SAS1R).

The antigenic fragments of the proteins of the invention may include peptide antigens that are at least about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150 or up to about 200 amino acids in length. Also included are full-length unprocessed protein as well as mature processed protein. These various length antigenic fragments may be designed in tandem order of linear amino acid sequence of the immunogen of choice, such as SAS1R, or staggered in linear sequence of the protein. In addition, antibodies to three-dimensional epitopes, i.e., non-linear epitopes, can also be prepared, based on, e.g., crystallographic data of proteins. Hosts may also be injected with peptides of different lengths encompassing a desired target sequence. Antibodies obtained from that injection may be screened against the short antigens of SAS1R and against mature SAS1R. Antibodies prepared against a SAS1R peptide may be tested for activity against that peptide as well as the full length SAS1R protein. Antibodies may have affinities of at least about 10⁻⁶M, 10⁻⁷M, 10⁻⁸M, 10⁻⁹M, 10⁻¹⁰M, 10⁻¹¹M or 10⁻¹²M toward the SAS1R peptide and/or the full-length SAS1R protein.

In one embodiment, the invention provides a therapeutic cancer vaccine comprising a pharmaceutical composition of the invention, said composition comprising one or more proteins, or variants, homologs, or fragments thereof, comprising an amino acid sequence selected from the group consisting of SEQ ID NOs: 6, 8, 10, 19, 20, 21, and 23, and fragments and homologs thereof, and optionally at least one other egg protein, or a variant, fragment, or homolog thereof.

Because of the temporally regulated expression of SAS1R in normal cells, any cells that might be killed would not include the early stage germ cells.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R Liss, Inc., pp. 77-96) may be employed to produce human monoclonal antibodies. In another embodiment, monoclonal antibodies are produced in germ-free animals.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Bio1.248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method that utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄ -, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

Despite their high selectivity, mAbs are frequently unable to mediate the killing of all the malignant cells. Indeed, differential surface marker expression and variable cell surface phenotypes can be observed in patients with the same type of hematological neoplasia. Polyclonal antibodies, which bind several antigens on a wide range of cells, offer the great advantage of preventing the escape of neoplastic clones during immunotherapy. Therefore, the present invention encompasses the use of not just mAbs, but also polyclonal antibodies (Polito et al., 2011, Toxins, 3:697-720).

B. Cytotoxic Agent

A cytotoxic agent can be any agent that can be delivered to a cell and induce cell death or inhibition. An example of such agents is ribosome inactivating proteins. A ribosome inactivating protein is a protein synthesis inhibitor that acts at the ribosome. A number of bacterial and plant toxins act by inhibiting protein synthesis in eukaryotic cells. The toxins of the Shiga and ricin family inactivate 60S ribosomal subunits by an N-glycosidic cleavage which releases a specific adenine base from the sugar-phosphate backbone of 28S rRNA. Members of the family include shiga and shiga0like toxins and type I (e.g., trichosanthin and luffin) and type II (e.g., ricin, agglutinin and abrin) ribosome inactivating proteins (RIPs). All these toxins are structurally related.

Saporin: Saporin is a highly toxic, ribosome inactivating protein (30 kDa) with N-glycosidase activity, from the seeds of Saponaria officinalis (common name: soapwart). Saporin acts by removing a single adenine base from position 4324 in 28S ribosomal RNA within the 60S ribosomal subunit. The removal of this base inhibits the ability of the ribosome to participate in protein synthesis. Saporin exhibits unusual stability due to its ability to resist denaturation and proteolysis. Saporin is relatively safe to work with because it is far less toxic than other such molecules, as Saporin lacks a domain for inserting itself into cells. However, saporin is highly toxic if given a means to gain entry into cells. Thus, the present invention includes antibodies conjugated to saporin. Saporin (GenBank: CAA41948.1; embl accession X59255.1) has the following sequence:

MKIYVVATIAWILLQFSAWTTTDAVTSITLDLVNPTAGQYSSFVDKI
RNNVKDPNLKYGGTDIAVIGPPS
KDKFLRINFQSSRGTVSLGLKRDNLYVVAYLAMDNTNVNRAYYFKSE
ITSAELTALFPEATTANQKALEY
TEDYQSIEKNAQITQGDKSRKELGLGIDLLLTFMEAVNKKARVVKNE
ARFLLIAIQMTAEVARFRYIQNL
VTKNFPNKFDSDNKVIQFEVSWRKISTAIYGDAKNGVFNKDYDFGFG
KVRQVKDLQMGLLMYLGKPKSSN
EANSTAYATTVL.

The term “ricin” includes reference to the lectin RCA₆₀ from Ricinus communis (Castor bean). The term also references toxic variants thereof. See, U.S. Pat. Nos. 5,079,163 and 4,689,401. Ricinus communis agglutinin (RCA) occurs in two forms designated RCA₆₀ and RCA₁₂₀ according to their molecular weights of approximately 65,000 and 120,000, respectively. Nicholson and Blaustein, J. Biochim. Biophys. Acta, 266:543 (1972). RCA₆₀, also referred to as RCA_{II′}, Ricin D or RCL III is extremely toxic, inhibits protein synthesis and has an affinity for N-acetyl-D-galactosamine. The toxin is a dimer of an A-chain (30,000 Da) and B-chain (33,000 Da) joined by a disulfide bond. The A chain is responsible for inactivating protein synthesis and killing cells. The B chain binds ricin to cell-surface galactose residues and facilitates transport of the A chain into the cytosol (Olsnes et al., Nature, 1974; 249:627-631). See, U.S. Pat. No. 3,060,165.

The term “abrin” includes reference to the toxic lectins from Abrus precatorius. The toxic principles, abrin a, b, c, and d, have a molecular weight of from about 63,000 and 67,000 Da and are composed of two disulfide-linked polypeptide chains A and B. The A chain inhibits protein synthesis; the B-chain (abrin-b) binds to D-galactose residues. See, Funatsu et al, The amino acid sequence of the A-chain of abrin-a and comparison with ricin, Agr. Biol. Chem. 52:1095 (1988). See also, Olsnes, Methods Enzymol. 50:330-335 (1978).

C. Immunotoxins

Toxic enzymes from plants and bacteria such as ricin, diphtheria toxin and Pseudomonas toxin have been coupled to antibodies or receptor binding ligands to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)). Regardless of the fact that the cell-recognition moiety is not always an antibody, these directed toxins are generally known as immunotoxins. These hybrid proteins kill cells which express the receptor or cell surface marker that the antibody or ligand portion of the molecule recognizes.

Under appropriate conditions, depending on the particular receptor or cell marker, the toxin enters the cytosol, inactivates the protein synthesis machinery and causes death of the target cell. Immunotoxins, which have been shown to be highly cytotoxic to cancer cells growing in cell culture and in animal models, demonstrate the potential of these reagents to treat blood and lymph borne malignancies which, because of their dissemination are not treatable by traditional surgical techniques, as well as solid tumors in restricted compartments such as the intraperitoneal cavity (reviewed in Griffin, et al., IMMUNOTOXINS, p 433, Boston/Dordrecht/Lancaster, Kluwer Academic Publishers, (1988); Vitetta, et al., Science 238:1098 (1987); Fitzgerald, et al., J. Nat'l Cancer Inst. 81:1455 (1989)). Traditional chemotherapies, while being effective in the treatment of some cancerous conditions, exhibit undesired side effects due to the systemic toxicity of the chemotherapeutic compounds.

The toxic moiety and the antibody may be conjugated by chemical or by recombinant means (see, Rybak, et al., Tumor Targeting 1:141 (1995)). Chemical modifications include, for example, derivitization for the purpose of linking the moieties to each other, either directly or through a linking compound (e.g., linkers, including bifunctional linkers, are small chemical molecules that link cytotoxic agents to mAb; such as dimethyl hydrazide, acid labile hydrazine linkers, disulfide linkers, glucuronide sugar linkers), by methods that are well known in the art of protein chemistry. For example, the means of linking the toxic moiety and the recognition moiety comprises a heterobifunctional coupling reagent which ultimately contributes to formation of an intermolecular disulfide bond between the two moieties. Other types of coupling reagents that are useful in this capacity for the present invention are described, for example, in U.S. Pat. No. 4,545,985. Alternatively, an intermolecular disulfide may conveniently be formed between cysteines in each moiety which occur naturally or are inserted by genetic engineering. The means of linking moieties may also use thioether linkages between heterobifunctional crosslinking reagents or specific low pH cleavable crosslinkers or specific protease cleavable linkers or other cleavable or noncleavable chemical linkages. The means of linking moieties of the immunotoxins may also comprise a peptidyl bond formed between moieties which are separately synthesized by standard peptide synthesis chemistry or recombinant means.

Many procedures and linker molecules for attachment of various compounds including toxins are known. See, for example, European Patent Application No. 188,256; U.S. Pat. Nos. 4,671,958, 4,659,839, 4,414,148, 4,699,784; 4,680,338; 4,569,789; 4,589,071; and Borlinghaus et al. Cancer Res. 47: 4071-4075 (1987), which are incorporated herein by reference. In particular, production of various immunotoxin conjugates is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982), Waldmann, Science, 252: 1657 (1991), U.S. Pat. Nos. 4,545,985 and 4,894,443 which are incorporated herein by reference. See also, e.g., Birch and Lennox, Monoclonal Antibodies: Principles and Applications, Chapter 4, Wiley-Liss, New York, N.Y. (1995); U.S. Pat. Nos. 5,218,112, 5,090,914; Hermanson, Bioconjugate Techniques, Academic Press, San Diego, Calif. (1996). In preferred embodiments, the linker molecule is m-Malimidobenzoyl-N-hydroxysuccinimideester (MBS) which can be used to prepare immunotoxin conjugates as described, for example, in Youle and Nevelle, Proc. Natl. Acad. Sci., 77(9):5483-5486 (1980).

In some circumstances, it is desirable to free the toxin from the antibody when the immunotoxic conjugate has reached its target site. Therefore, immunotoxic conjugates comprising linkages which are cleavable in the vicinity or within the target site may be used when the toxin is to be released at the target site. Cleaving of the linkage to release the agent from the ligand may be prompted by enzymatic activity or conditions to which the immunoconjugate is subjected either inside the target cell or in the vicinity of the target site. A number of different cleavable linkers are known to those of skill in the art. See U.S. Pat. Nos. 4,618,492; 4,542,225, and 4,625,014. SPDP is a reversible NHS-ester, pyridyl disulfide cross-linker used to conjugate amine-containing molecules to sulfhydryls. Another chemical modification reagent is 2-iminothiolane which reacts with amines and yields a sulfhydryl. Water soluble SPDP analogs, such as Sulfo-LC-SPDP (Pierce, Rockford, Ill.) are also available. SMPT is a reversible NHS-ester, pyridyl disulfide cross-linker developed to avoid cleavage in vivo prior to reaching the antigenic target. Additionally, the NHS-ester of SMPT is relatively stable in aqueous solutions.

Possible chemical modifications of the protein moieties of the present invention also include derivitization with polyethylene glycol (PEG) or other polymers (such as dextran) to extend time of residence in the circulatory system and reduce immunogenicity, according to well known methods (See for example, Lisi, et al., Applied Biochem. 4:19 (1982); Beauchamp, et al., Anal Biochem. 131:25 (1982); and Goodson, et al., Bio/Technology 8:343 (1990)).

Possible genetic engineering modifications of the proteins of the immunotoxins include combination of the relevant functional domains of each into a single chain multi-functional biosynthetic protein expressed from a single gene derived by recombinant DNA techniques. (See, for example, PCT published application WO/88/09344). Furthermore, recombinant DNA techniques can be used to link the recombinant protein and the antibody. Accordingly, the immunotoxin can comprise a fused protein beginning at one end with the protein and ending with the antibody.

Methods of producing recombinant fusion proteins are well known to those of skill in the art. Thus, for example, Chaudhary, et al., Nature 339:394 (1989); Batra, et al., J. Biol. Chem. 265:15198 (1990); Batra, et al., Proc. Nat'l Acad. Sci. USA 86:8545 (1989); Chaudhary, et al., Proc. Nat'l Acad. Sci. USA 87:1066 (1990), all incorporated by reference, describe the preparation of various single chain antibody-toxin fusion proteins.

In general, producing immunotoxin fusion proteins involves separately preparing the F, light and heavy chains and DNA encoding the one protein to be used. The two sequences are combined in a plasmid or other vector to form a construct encoding the particular desired fusion protein. A simpler approach involves inserting the DNA encoding the particular F, region into a construct already encoding the desired one protein.

The invention includes nucleic acid constructs that encodes the novel proteins described here. A nucleic acid construct is one which, when incorporated into an appropriate vector, is capable of replicating in a host. The constructs may be linked to other sequences capable of affecting the expression of the construct, such as promoters and enhancers.

In one embodiment, Fab-ZAP mouse (Advanced Targeting Systems Catalog #IT-48) is used. It is a chemical conjugate of goat anti-mouse monovalent antibody and the ribosome-inactivating protein, saporin. Second immunotoxins are conjugations of a secondary antibody to saporin. Fab-ZAP uses a primary mouse monoclonal IgG antibody to target and eliminate cells that recognize the primary antibody. Once Fab-ZAP is internalized, saporin breaks away from the targeting agent and inactivates the ribosomes, which causes protein inhibition and, ultimately, cell death. Fab-ZAP is made with a monovalent secondary antibody eliminating the possibility of cap formation, while preserving all the qualities that make an effective in vitro diagnostic tool. It also has an improved ED₅₀ when directly compared to Mab-ZAP in a cytotoxicity assay. This reagent can be utilized for screening mouse IgG antibodies for internalization and/or their suitability to make potent immunotoxins.

D. Vaccines and Immunogens

In one embodiment, the invention relates to methods and reagents for immunizing and treating a subject with an antigenic compound of the invention such as SAS1R and fragments and homologs thereof, to elicit specific cellular and humoral immune-responses against such specific antigens. The invention provides methods of using specifically prepared immunogen in fresh or lyophilized liposome, proper routes of administration of the immunogen, proper doses of the immunogen, and specific combinations of heterologous immunization including DNA priming in one administration route followed by liposome-mediated protein antigen boost in a different route to tailor the immune responses in respects of enhancing cell mediated immune response, cytokine secretion, humoral immune response, especially skewing T helper responses to be Th1 or a balanced Th1 and Th2 type. For more detail, see Klinefelter (U.S. patent application Ser. No. 11/572,453, which claims priority to international patent application PCT/US2005/026102).

A homolog herein is understood to comprise an immunogenic polypeptide having at least 70%, preferably at least 80%, more preferably at least 90%, still more preferably at least 95%, still more preferably at least 98% and most preferably at least 99% amino acid sequence identity with the naturally occurring SAS1R polypeptides mentioned above and is still capable of eliciting at least the immune response obtainable thereby. A homolog or analog may herein comprise substitutions, insertions, deletions, additional N- or C-terminal amino acids, and/or additional chemical moieties, such as carbohydrates, to increase stability, solubility, and immunogenicity.

In one embodiment of the invention, the present immunogenic polypeptides as defined herein, are glycosylated. Without wishing to be bound by any particular theory, it is hypothesized herein that by glycosylation of these polypeptides the immunogenicity thereof may be increased. Therefore, in one embodiment, the aforementioned immunogenic polypeptide as defined herein before, is glycosylated, having a carbohydrate content varying from 10-80 wt %, based on the total weight of the glycoprotein or glycosylated polypeptide. More preferably said carbohydrate content ranges from 15-70 wt %, still more preferably from 20-60 wt %. In another embodiment, said glycosylated immunogenic polypeptide comprises a glycosylation pattern that is similar to that of the corresponding zona pellucida glycoprotein (or fragment thereof) of the human that is treated. It is hypothesized that this even further increases the immunogenicity of said polypeptide. Thus, it is preferred that the immunogenic polypeptide comprises a glycosylation pattern that is similar to that of the corresponding SAS1R glycoprotein.

In one embodiment, the source of a polypeptide comprises an effective amount of an immunogenic polypeptide selected from SAS1R protein, and immunologically active homologs thereof and fragments thereof, or a nucleic acid sequence encoding said immunogenic polypeptide.

In one embodiment, the present method of immunization comprises the administration of a source of immunogenically active polypeptide fragments, said polypeptide fragments being selected from SAS1R protein fragments and/or homologs thereof as defined herein before, said polypeptide fragments comprising dominant CTL and/or HTL epitopes and which fragments are between 18 and 45 amino acids in length. Peptides having a length between 18 and 45 amino acids have been observed to provide superior immunogenic properties as is described in WO 02/070006.

Peptides may advantageously be chemically synthesized and may optionally be (partially) overlapping and/or may also be ligated to other molecules, peptides, or proteins. Peptides may also be fused to form synthetic proteins, as in Welters et al. (Vaccine. 2004 Dec. 2; 23(3):305-11). It may also be advantageous to add to the amino- or carboxy-terminus of the peptide chemical moieties or additional (modified or D-) amino acids in order to increase the stability and/or decrease the biodegradability of the peptide. To improve immunogenicity, immuno-stimulating moieties may be attached, e.g. by lipidation or glycosylation. To enhance the solubility of the peptide, addition of charged or polar amino acids may be used, in order to enhance solubility and increase stability in vivo.

For immunization purposes, the aforementioned immunogenic polypeptides of the invention may also be fused with proteins, such as, but not limited to, tetanus toxin/toxoid, diphtheria toxin/toxoid or other carrier molecules. The polypeptides according to the invention may also be advantageously fused to heatshock proteins, such as recombinant endogenous (murine) gp96 (GRP94) as a carrier for immunodominant peptides as described in (references: Rapp U K and Kaufmann S H, Int Immunol. 2004 April; 16(4):597-605; Zugel U, Infect Immun. 2001 June; 69(6):4164-7) or fusion proteins with Hsp70 (Triebel et al; WO9954464).

The individual amino acid residues of the present immunogenic (poly)peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the alpha carbon, amide carbonyl, complete replacement of the amide bond, extensions, deletions, or backbone cross-links See, generally, Spatola, Chemistry and Biochemistry of Amino Acids, Peptides and Proteins, Vol. VII (Weinstein ed., 1983). Several peptide backbone modifications are known and can be used in the practice of the invention.

Amino acid mimetics may also be incorporated in the polypeptides. An “amino acid mimetic” as used here is a moiety other than a naturally occurring amino acid that conformationally and functionally serves as a substitute for an amino acid in a polypeptide of the present invention. Such a moiety serves as a substitute for an amino acid residue if it does not interfere with the ability of the peptide to elicit an immune response against the native SAS1R T cell epitopes. Amino acid mimetics may include non-protein amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

In one embodiment, the present method comprises the administration of a composition comprising one or more of the present immunogenic polypeptides as defined herein above, and at least one excipient. Excipients are well known in the art of pharmacy and may for instance be found in textbooks such as Remington's pharmaceutical sciences, Mack Publishing, 1995.

The present method for immunization may further comprise the administration, and in one aspect, the co-administration, of at least one adjuvant. Adjuvants may comprise any adjuvant known in the art of vaccination and may be selected using textbooks like Current Protocols in Immunology, Wiley Interscience, 2004.

Adjuvants are herein intended to include any substance or compound that, when used, in combination with an antigen, to immunize a human or an animal, stimulates the immune system, thereby provoking, enhancing or facilitating the immune response against the antigen, preferably without generating a specific immune response to the adjuvant itself. In one aspect, adjuvants can enhance the immune response against a given antigen by at least a factor of 1.5, 2, 2.5, 5, 10, or 20, as compared to the immune response generated against the antigen under the same conditions but in the absence of the adjuvant. Tests for determining the statistical average enhancement of the immune response against a given antigen as produced by an adjuvant in a group of animals or humans over a corresponding control group are available in the art. The adjuvant preferably is capable of enhancing the immune response against at least two different antigens. The adjuvant of the invention will usually be a compound that is foreign to a human, thereby excluding immunostimulatory compounds that are endogenous to humans, such as e.g. interleukins, interferons, and other hormones.

A number of adjuvants are well known to one of ordinary skill in the art. Suitable adjuvants include, e.g., incomplete Freund's adjuvant, alum, aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dip-almitoyl-sn-glycero-3-hydroxy-phosphoryloxy)-ethylamine (CGP 19835A, referred to as MTP-PE), DDA (2 dimethyldioctadecylammonium bromide), polyIC, Poly-A-poly-U, RIBI™, GERBU™, Pam3™, Carbopol™, Specol™, Titermax™, tetanus toxoid, diphtheria toxoid, meningococcal outer membrane proteins, diphtheria protein CRM₁₉₇. Preferred adjuvants comprise a ligand that is recognized by a Toll-like-receptor (TLR) present on antigen presenting cells. Various ligands recognized by TLR's are known in the art and include e.g. lipopeptides (see, e.g., WO 04/110486), lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications.

The methods of immunization of the present application further encompass the administration, including the co-administration, of a CD40 binding molecule in order to enhance a CTL response and thereby enhance the therapeutic effects of the methods and compositions of the invention. The use of CD40 binding molecules is described in WO 99/61065, incorporated herein by reference. The CD40 binding molecule is preferably an antibody or fragment thereof or a CD40 Ligand or a variant thereof, and may be added separately or may be comprised within a composition according to the current invention. Such effective dosages will depend on a variety of factors including the condition and general state of health of the patient. Thus, dosage regimens can be determined and adjusted by trained medical personnel to provide the optimum therapeutic or prophylactic effect.

In the present method, the one or more immunogenic polypeptides are typically administered at a dosage of about 1 ug/kg patient body weight or more at least once. Often dosages are greater than 10 ug/kg. According to the present invention, the dosages preferably range from 1 ug/kg to 1 mg/kg.

In one embodiment typical dosage regimens comprise administering a dosage of 1-1000 ug/kg, more preferably 10-500 ug/kg, still more preferably 10-150 ug/kg, once, twice or three times a week for a period of one, two, three, four or five weeks. According to one embodiment, 10-100 ug/kg is administered once a week for a period of one or two weeks.

The present method, in one aspect, comprises administration of the present immunogenic polypeptides and compositions comprising them via the injection, transdermal, or oral route. In another, embodiment of the invention, the present method comprises vaginal administration of the present immunogenic polypeptides and compositions comprising them.

Another aspect of the invention relates to a pharmaceutical preparation comprising as the active ingredient the present source of a polypeptide as defined herein before. More particularly pharmaceutical preparation comprises as the active ingredient one or more of the aforementioned immunogenic polypeptides selected from the group of SAS1R proteins, homologues thereof and fragments of said SAS1R proteins and homologs thereof, or, alternatively, a gene therapy vector as defined herein above.

The present invention further provides a pharmaceutical preparation comprising one or more of the immunogenic polypeptides of the invention. The concentration of said polypeptide in the pharmaceutical composition can vary widely, i.e., from less than about 0.1% by weight, usually being at least about 1% by weight to as much as 20% by weight or more.

The composition may comprise a pharmaceutically acceptable carrier in addition to the active ingredient. The pharmaceutical carrier can be any compatible, non-toxic substance suitable to deliver the immunogenic polypeptides or gene therapy vectors to the patient. For polypeptides, sterile water, alcohol, fats, waxes, and inert solids may be used as the carrier. Pharmaceutically acceptable adjuvants, buffering agents, dispersing agents, and the like, may also be incorporated into the pharmaceutical compositions.

In one embodiment, the present pharmaceutical composition comprises an adjuvant, as defined in more detail herein before. Adjuvants useful for incorporation in the present composition are preferably selected from the group of ligands that are recognized by a Toll-like-receptor (TLR) present on antigen presenting cells, including lipopeptides, lipopolysaccharides, peptidoglycans, liopteichoic acids, lipoarabinomannans, lipoproteins (from mycoplasma or spirochetes), double-stranded RNA (poly I:C), unmethylated DNA, flagellin, CpG-containing DNA, and imidazoquinolines, as well derivatives of these ligands having chemical modifications. The routineer will be able to determine the exact amounts of anyone of these adjuvants to be incorporated in the present pharmaceutical preparations in order to render them sufficiently immunogenic. According to another preferred embodiment, the present pharmaceutical preparation may comprise one or more additional ingredients that are used to enhance CTL immunity as explained herein before. According to a particularly preferred embodiment the present pharmaceutical preparation comprises a CD40 binding molecule.

Methods of producing pharmaceutical compositions comprising polypeptides are described in U.S. Pat. Nos. 5,789,543 and 6,207,718. The preferred form depends on the intended mode of administration and therapeutic application.

In one embodiment, the present immunogenic proteins or polypeptides are administered by injection. The parenteral route for administration of the polypeptide is in accordance with known methods, e.g. injection or infusion by intravenous, intraperitoneal, intramuscular, intra-arterial, subcutaneous, or intralesional routes. The protein or polypeptide may be administered continuously by infusion or by bolus injection. A typical composition for intravenous infusion could be made up to contain 10 to 50 ml of sterile 0.9% NaCl or 5% glucose optionally supplemented with a 20% albumin solution and between 10 ug and 50 mg, preferably between 50 ug and 10 mg, of the polypeptide. A typical pharmaceutical composition for intramuscular injection would be made up to contain, for example, 1-10 ml of sterile buffered water and between 10 ug and 50 mg, preferably between 50 ug and 10 mg, of the polypeptide of the present invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

For convenience, immune responses are often described in the present invention as being either “primary” or “secondary” immune responses. A primary immune response, which is also described as a “protective” immune response, refers to an immune response produced in an individual as a result of some initial exposure (e.g., the initial “immunization”) to a particular antigen. Such an immunization can occur, for example, as the result of some natural exposure to the antigen (for example, from initial infection by some pathogen that exhibits or presents the antigen). Alternatively, the immunization can occur because of vaccinating the individual with a vaccine containing the antigen. For example, the vaccine can be a vaccine comprising one or more antigenic epitopes or fragments of SAS1R.

The vaccine can also be modified to express other immune activators such as IL2, and costimulatory molecules, among others.

Another type of vaccine that can be combined with antibodies to an antigen is a vaccine prepared from a cell lysate of interest, in conjunction with an immunological adjuvant, or a mixture of lysates from cells of interest plus DETOX™ immunological adjuvant. Vaccine treatment can be boosted with anti-antigen antibodies, with or without additional chemotherapeutic treatment.

When used in vivo for therapy, the antibodies of the subject invention are administered to the subject in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks. Optionally, the administration is made during the course of adjunct therapy such as antimicrobial treatment, or administration of tumor necrosis factor, interferon, or other cytoprotective or immunomodulatory agent.

For parenteral administration, the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1.0 mg/ml to about 10 mg/ml.

Use of IgM antibodies can be preferred for certain applications; however, IgG molecules by being smaller can be more able than IgM molecules to localize to certain types of infected cells.

There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize. Therefore, antigen-antibody combinations of the type specified by this invention can be used in many ways. Additionally, purified antigens (Hakomori, Ann. Rev. Immunol. 2:103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human patients.

The antibody compositions used are formulated and dosages established in a fashion consistent with good medical practice taking into account the condition or disorder to be treated, the condition of the individual patient, the site of delivery of the composition, the method of administration, and other factors known to practitioners. The antibody compositions are prepared for administration according to the description of preparation of polypeptides for administration, infra.

As is well understood in the art, biospecific capture reagents include antibodies, binding fragments of antibodies which bind to activated integrin receptors on metastatic cells (e.g., single chain antibodies, Fab′ fragments, F(ab)′2 fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

The hybrid antibodies and hybrid antibody fragments include complete antibody molecules having full length heavy and light chains, or any fragment thereof, such as Fab, Fab′, F(ab′)2, Fd, scFv, antibody light chains and antibody heavy chains. Chimeric antibodies which have variable regions as described herein and constant regions from various species are also suitable. See for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, RNAi molecules, or ribozymes, aptamers, peptides or low-molecular weight organic compounds recognizing said polynucleotides or polypeptides, as well as the protein itself and fragments thereof.

The present invention further encompasses the identification of functional fragments for the use of SAS1R for use as antigens for therapeutic antibodies as well as its use as an immunogen and as an anticancer vaccine.

In one embodiment, a mimotope analysis of full length SAS1R can be performed by subdividing the sequence into, for example, a series of 15 amino acid peptides, with each peptide overlapping by three amino acids. All peptides can be biotinylated and allowed to bind to streptavidin-coated wells in 96-well plates. The reactivity of various antisera can be detected by enzyme-linked immunosorbent assay (ELISA). After blocking non-specific binding, SAS1R antibody can be added sequentially (i.e., either affinity-purified anti-SAS1R or affinity-purified anti-full-length recombinant SAS1R), followed by the sequential addition of peroxidase-conjugated secondary antibody, and peroxidase substrate.

The optical density of each well can be read at 450 nm and duplicate wells averaged. The average value obtained from a similar ELISA using control serum (i.e., preimmune serum) can be subtracted from the test Ig values and the resultant values plotted to determine which linear epitopes are recognized by the Ig.

The second and third components in the strategy to identify functional fragments of SAS1R rely on the synthesis of non-biotinylated peptides corresponding to the epitopes (peptides) predicted by the mimotope analysis. To determine whether any of the epitopes recognized by mimotope analysis are exposed on the egg, immunocytochemical staining with the Ig, without and with each of the peptides, can performed.

E. Pharmaceutical Compositions

The present invention also relates to a pharmaceutical composition comprising immunotoxins of the present invention in a pharmaceutically acceptable carrier. In therapeutic applications, compositions are administered to a patient suffering from a disease, in an amount sufficient to cure or at least partially arrest the disease and its complications. An amount adequate to accomplish this is defined as a therapeutically effective dose. Amounts effective for this use will depend on the severity of the disease and the general state of the patient's health.

Advantageously, the pharmaceutical composition is suitable for parenteral administration. The immunotoxins of the present invention may be administered by various means appropriate for different purposes, for example, for treating tumors in various parts of the body, according to methods known in the art for other immunotoxins. (See, for example, Rybak, et al., Human Cancer Immunology, in IMMUNOLOGY AND ALLERGY CLINICS OF AMERICA, W. B. Saunders, 1990, and references cited therein). Accordingly, the present invention also relates to pharmaceutical compositions comprising an immunotoxin of this invention and a pharmaceutically acceptable carrier, particularly such compositions which are suitable for the above means of administration.

Single or multiple administrations of the compositions may be administered depending on the dosage and frequency as required and tolerated by the patient. In any event, the composition should provide a sufficient quantity of the proteins of this invention to effectively treat the patient.

The compositions for administration will commonly comprise a solution of the fusion protein comprising the single chain antibody and the toxin dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of fusion protein in these formulations can vary widely, and will be selected primarily based on fluid volumes, viscosities, body weight and the like in accordance with the particular mode of administration selected and the patient's needs.

The immunotoxins of this invention may be administered systemically by injection, such as intravenously, but also intramuscularly, subcutaneously, intrathecally, intraperitoneally, into vascular spaces, or into joints, e.g., intraarticular injection. The dose will be dependent upon the properties of the immunotoxin employed, e.g., its activity and biological half-life, the concentration of the immunotoxin in the formulation, the site and rate of dosage, the clinical tolerance of the patient involved, the extent of cancer afflicting the patient and the like as is well within the skill of the physician.

Administration may also be intranasal or by other nonparenteral routes. The immunotoxin may also be administered via microspheres, liposomes or other microparticulate delivery systems placed in certain tissues including blood.

The immunotoxin of the present invention may be administered in solution. The pH of the solution can be in the range of pH 5 to 9.5, such as pH 6.5 to 7.5. The immunotoxins or derivatives thereof can be in a solution having a suitable pharmaceutically acceptable buffer such as phosphate, tris(hydroxymethyl)aminomethane-HCl or citrate and the like. Buffer concentrations should be in the range of 1 to 100 mM. The solution of the immunoglobulin can also contain a salt, such as sodium chloride or potassium chloride in a concentration of 50 to 1 50 mM. An effective amount of a stabilizing agent such as albumin, a globulin, a detergent, a gelatin, a protamine or a salt of protamine can also be included and can be added to a solution containing the immunotoxin or to the composition from which the solution is prepared. The immunotoxin may be formulated with a polymer (such as polyethylene glycol (PEG) or dextran), which can be used to increase the biological half-life of the immunotoxin, thus resulting in a more extended period of activity. Systemic administration of the immunotoxin can be made every two to three days or once a week if a humanized or human form of the antibody is used. Alternatively, daily administration is useful.

Thus, a typical pharmaceutical composition for intravenous administration would be about 0.01 to 100 mg per patient per day. Dosages from 0.1 up to about 1000 mg per patient per day may be used, particularly when the drug is administered to a secluded site and not into the blood stream, such as into a tumor or an organ within which a tumor resides. Actual methods for preparing parenterally administrable compositions will be known or apparent to those skilled in the art and are described in more detail in such publications as REMINGTON'S PHARMACEUTICAL SCIENCE, 15TH ED., Mack Publishing Co., Easton, Pa., (1980).

Further, the present invention relates to a method of selectively killing cells using a selective immunotoxin of the present invention having an antibody specific for a target on the surface or intracellular target of the cells to be killed under conditions allowing binding of the antibody. Binding of the antibody to the surface or intracellular marker on or in a cell causes the toxin portion of the reagent to selectively kill the cell.

The present invention is also directed to pharmaceutical compositions comprising the compounds of the present invention. More particularly, such compounds can be formulated as pharmaceutical compositions using standard pharmaceutically acceptable carriers, fillers, solublizing agents and stabilizers known to those skilled in the art.

The invention is also directed to methods of administering the compounds of the invention to a subject. In one embodiment, the invention provides a method of treating a subject by administering compounds identified using the methods of the invention description. Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a subject in need of such treatment is provided. The method comprises administering a pharmaceutical composition comprising at least one compound of the present invention to a subject in need thereof. Compounds identified by the methods of the invention can be administered with known compounds or other medications as well.

The invention also encompasses the use of pharmaceutical compositions of an appropriate compound, and homologs, fragments, analogs, or derivatives thereof to practice the methods of the invention, the composition comprising at least one appropriate compound, and homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. The invention is also contemplated for use in contraception for nuisance animals such as rodents.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the condition or disease being treated, the type and age of the animal, etc.

Suitable preparations of vaccines include injectables, either as liquid solutions or suspensions, however, solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

The invention is also directed to methods of administering the compounds of the invention to a subject. In one embodiment, the invention provides a method of treating a subject by administering compounds identified using the methods of the invention. Pharmaceutical compositions comprising the present compounds are administered to an individual in need thereof by any number of routes including, but not limited to, topical, oral, intravenous, intramuscular, intra arterial, intramedullary, intrathecal, intraventricular, transdermal, subcutaneous, intraperitoneal, intranasal, enteral, topical, sublingual, or rectal means.

In accordance with one embodiment, a method of treating and vaccinating a subject in need of such treatment is provided. The method comprises administering a pharmaceutical composition comprising at least one compound of the present invention to a subject in need thereof. Compounds identified by the methods of the invention can be administered with known compounds or other medications as well.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate, and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

A variety of vaginal drug delivery systems is known in the art. Suitable systems include creams, foams, tablets, gels, liquid dosage forms, suppositories, and pessaries. Mucoadhesive gels and hydrogels, comprising weakly crosslinked polymers which are able to swell in contact with water and spread onto the surface of the mucosa, have been used for vaccination with peptides and proteins through the vaginal route previously. The present invention further provides for the use of microspheres for the vaginal delivery of peptide and protein drugs. More detailed specifications of vaginally administered dosage forms including excipients and actual methods of preparing said dosage forms are known, or will be apparent, to those skilled in this art. For example, Remington's Pharmaceutical Sciences (15th ed., Mack Publishing, Easton, Pa., 1980) is referred to.

The invention also includes a kit comprising the composition of the invention and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

Other techniques known in the art may be used in the practice of the present invention, including those described in international patent application WO 2006/091535 (PCT/US2006/005970), the entirety of which is incorporated by reference herein.

Peptide Modification and Preparation

It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or non-standard synthetic amino acids. The peptides of the invention are not limited to products of any of the specific exemplary processes listed herein.

The invention includes the use of beta-alanine (also referred to as β-alanine, β-Ala, bA, and βA, having the structure:

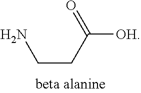

Sequences are provided herein which use the symbol “βA”, but in the Sequence Listing submitted herewith “βA” is provided as “Xaa” and reference in the text of the Sequence Listing indicates that Xaa is beta alanine.

Peptides useful in the present invention, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenly esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C₄ -, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the invention are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of Alkyl-Substituted Hydrophobic Amino Acids:

including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of Aromatic-Substituted Hydrophobic Amino Acids:

including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′, -3′, - or 4′-methyl-2, 3 or 4-biphenylalanine, and 2- or 3-pyridylalanine.

Substitution of Amino Acids Containing Basic Functions:

including arginine, lysine, histidine, ornithine, 2,3-diaminopropionic acid, homoarginine, alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of Acidic Amino Acids:

including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of Side Chain Amide Residues:

including asparagine, glutamine, and alkyl or aromatic substituted derivatives of asparagine or glutamine.

Substitution of Hydroxyl Containing Amino Acids:

including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e.g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gln, asn, lys; Asn (N) his, asp, lys, arg, gln; Asp (D) asn, glu; Cys (C) ala, ser; Gln (Q) glu, asn; Glu (E) gln, asp; Gly (G) ala; His (H) asn, gln, lys, arg; Ile (I) val, met, ala, phe, leu; Leu (L) val, met, ala, phe, ile; Lys (K) gln, asn, arg; Met (M) phe, ile, leu; Phe (F) leu, val, ile, ala, tyr; Pro (P) ala; Ser (S), thr; Thr (T) ser; Trp (W) phe, tyr; Tyr (Y) trp, phe, thr, ser; Val (V) ile, leu, met, phe, ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See, e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. (Id.) Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Linkers

Additionally, modifications encompassed by the invention include introduction of linkers or spacers between the targeting sequence of the binding moiety or binding polypeptide and the detectable label or therapeutic agent. For example, use of such linkers/spacers can improve the relevant properties of the binding peptides (e.g., increase serum stability, etc.). These linkers can include, but are not restricted to, substituted or unsubstituted alkyl chains, polyethylene glycol derivatives, amino acid spacers, sugars, or aliphatic or aromatic spacers common in the art.

For example, suitable linkers include homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Some examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. N-succinimidyl 3-(2-pyridyldithio)propionate is preferred. Some examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Furthermore, linkers that are combinations of the molecules and/or moieties described above, can also be employed to confer special advantage to the properties of the peptide. Lipid molecules with linkers may be attached to allow formulation of ultrasound bubbles, liposomes or other aggregation based constructs. Such constructs could be employed as agents for targeting and delivery of a diagnostic reporter, a therapeutic agent (e.g., a chemical “warhead” for therapy), or a combination of these.

Constructs employing dimers, multimers, or polymers of one or more peptide ligands of the invention are also contemplated. Indeed, there is ample literature evidence that the binding of low potency peptides or small molecules can be substantially increased by the formation of dimers and multimers. Thus, dimeric and multimeric constructs (both homogeneous and heterogeneous) are within the scope of the instant invention. The polypeptide sequences in the dimeric constructs can be attached at their N- or C-terminus or the N-epsilon nitrogen of a suitably placed lysine moiety (or another function bearing a selectively derivatizable group such as a pendant oxyamino or other nucleophilic group), or can be joined together via one or more linkers (e.g., those discussed herein) employing the appropriate attachment chemistry. This coupling chemistry can include amide, urea, thiourea, oxime, or aminoacetylamide (from chloro- or bromoacetamide derivatives, but is not so limited). Linkers can also be used for attachment to a chelating agent.

Therapeutic Agents

In other embodiments, therapeutic agents, including, but not limited to, cytotoxic agents, anti-angiogenic agents, pro-apoptotic agents, antibiotics, hormones, hormone antagonists, chemokines, drugs, prodrugs, toxins, enzymes or other agents may be used as adjunct therapies when using the antibody/peptide ligand complexes described herein. Drugs useful in the invention may, for example, possess a pharmaceutical property selected from the group consisting of antimitotic, antikinase, alkylating, antimetabolite, antibiotic, alkaloid, anti-angiogenic, pro-apoptotic agents and combinations thereof.

In another aspect, the present invention provides methods for identifying a cancer cell-binding partner having selective affinity for SAS1R. The methods include selectively immobilizing a diverse population of binding molecules to a solid support, contacting (e.g., simultaneously contacting) the diverse population immobilized on the solid support with one or more SAS1R peptides or cells expressing SAS1R and determining at least one binding molecule which selectively binds to one or more of the SAS1R peptide ligands, including those expressed by a bacteriophage. Also described herein are rapid and efficient methods for the identification of binding molecules that exhibit selective affinity for one or more SAS1R binding molecules of interest. The methods are advantageous in that they allow the simultaneous screening of multiple binding molecules. Moreover, very little information is required regarding the identity or function of either the binding molecule or the ligand for use in the present inventions. For example, diverse populations of binding molecules can be simultaneously screened against diverse populations of peptide ligands to rapidly identify numerous molecules exhibiting a desired binding specificity. The methods described herein can therefore be advantageously applied for the discovery of specific reagents, such as peptide ligands and biomarkers, for diagnosis and treatment of human diseases.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

The invention is now described with reference to the following Examples and Embodiments. Without further description, it is believed that one of ordinary skill in the art can, using the preceding description and the following illustrative examples, make and utilize the present invention and practice the claimed methods. The following working examples therefore, are provided for the purpose of illustration only and specifically point out the preferred embodiments of the present invention, and are not to be construed as limiting in any way the remainder of the disclosure. Therefore, the examples should be construed to encompass any and all variations which become evident as a result of the teaching provided herein.

F. Cancer Diagnosis

Detection and diagnosis of SAS1R positive cancers can be performed by obtaining samples from a subject and determining whether the sample is positive for SAS1R and compositions and methods are also provided for in vivo imaging of SAS1R positive cells.

In one embodiment, tumors expressing SAS1R can be directly targeted for diagnosis. This can be done for example using antibodies or fragments thereof that are directed against SAS1R and which have been conjugated to an imaging agent useful for in vivo imaging.

In one embodiment, tissue samples and other samples obtained from a subject can be used to detect SAS1R. Tissue samples can include tumor biopsies and other tissues where SAS1R from cancer cells, including SAS1R shed from dead cancer cells. The samples other than tumor biopsies include, but are not limited to, tissue samples, blood, plasma, peritoneal fluids, ascites, follicular fluid, urine, feces, saliva, mucus, phlegm, sputum, tears, cerebrospinal fluid, effusions such as lung effusions, lavage, and Pap smears.

Antibodies and other peptides can be conjugated to a number of agents capable of being imaged in vivo and used for imaging/detection in ex vivo tests and assays such as immunofluorescence, ELISA, etc. In one embodiment, the antibody is detected using at least one of enzyme-linked immunoassay, western blot, lateral flow membrane test, latex agglutination, and other forms of immunochromatography or immunoassay utilizing at least one antibody. In one embodiment, SAS1R proteins are detected using ELISA.

Multiple techniques for measuring proteins and peptides are known in the art or described herein and can use in the practice of the invention. These include, but are not limited to, for example:

Electrochemiluminescent immunoassay;

Bioluminsescent Immunoassay (for example, with use of apoaequorin and oelenterazine);

Luminescent oxygen channeling immunoassay (LOCI);

The Erenna Immunoassay System (a modified microparticle-based sandwich immunoassay with single-molecule counting);

Nanoparticle Immunoassay: nano-particles, spheres, or tubes as solid phases
upconverting phosphor nanoparticle using antiStokes shift
quantum dot immunoassay (Heterogeneous immunoassay in which a nanometer-sized (less than 10 nm) semiconductor quantum dot is used as a label. A quantum dot is a highly fluorescent nanocrystal composed of CdSe, CdS, ZnSe, InP, or InAs or a layer of ZnS or CdS on, for example, a CdSe core);

Fluorescence Excitation Transfer Immunoassay;

ImmunoPCR Immunoassay;

Solid Phase, Light-Scattering Immunoassay: Indium spheres are coated on glass to measure an antibody binding to an antigen. Binding of antibodies to antigens increases dielectric layer thickness, which produces a greater degree of scatter than in areas where only an antigen is bound. Quantitation is achieved by densitometry; and

Surface Effect Immunoassay: with antibody immobilized on the surface of a waveguide (a quartz, glass, or plastic slide, or a gold- or silver-coated prism), and binding of antigen measured directly by total internal reflection fluorescence, surface plasmon resonance, or attenuated total reflection.

In one aspect, an antibody or a fragment or homolog thereof of the invention can be conjugated to an imaging agent. In one embodiment, antibody complex comprises an imaging agent selected from the group consisting of a radionuclide, a radiological contrast agent, a paramagnetic ion, a metal, a biological tag, a fluorescent label, a chemiluminescent label, an ultrasound contrast agent and a photoactive agent. In one aspect, the imaging agent is a radionuclide. In one aspect, the radionuclide is selected from the group consisting of ¹¹⁰In, ¹¹¹In, ¹⁷⁷Lu, ¹⁸F, ⁵²Fe, ⁶²cu, ⁶⁴cu, ⁶⁷cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹²⁰I, ¹²³I, ¹²⁴I, ¹²⁵I, ¹³¹I, ¹⁵⁴⁻¹⁵⁸Gd, ³²F, ¹¹C, ¹³N, ¹⁵O, ¹⁸⁶Re, ¹⁸⁸Re, ⁵¹Mn, ⁵²mMn, ³³Co, ⁷²As, ⁷⁵Br, ⁷⁶Br, ⁸²mRb, ⁸³Sr, and othergamma-, beta-, or positron-emitters. In one aspect, the radionuclide is ¹¹¹In.

The invention further provides for use of the monoclonal antibodies described herein for drug delivery and for diagnostics. For example, various agents as described herein can be conjugated to the antibodies. Drugs such as calicheamicin, peptides such as D(KLAKLAK)², and radionuclides such as beta ⁹⁰Y, gamma, ¹³¹I, and positron ¹²⁴I emitters can be conjugated to monoclonal antibodies to human SAS1R and used to image lung tumors, as radiotherapeutic and chemotherapeutic agents for treatment.

The invention further provides a method for detecting cancer, diagnosing cancer, monitoring the progression of cancer, or monitoring treatment of a cancer, wherein the cancer cells express or present SAS1R or a homolog or fragment thereof. The method comprises administering to a test subject a pharmaceutical composition comprising a peptide ligand complex wherein the complex comprises an imaging agent, and then detecting the imaging agent and determining the levels and location of the imaging agent in a test subject. The levels are determined using the systems, computers, and programs for that particular kind of imaging, whether it be MRI, PET, SPECT/CT, CAT scans, X-rays, ultrasound, etc. The values obtained from the final processing of the levels are then used for the comparison. A comparison of the levels and location in the test subject is made with the levels and location of the imaging agent from an otherwise identical location from an unaffected subject or with an unaffected area of the test subject. A higher level or different location of the imaging agent in the test subject compared with the level or location of the imaging agent in said sample from an unaffected subject or from an unaffected area of the test subject, is an indication that the test subject has a cancer expressing or presenting SAS1R or a homolog or fragment thereof. The levels or location of the detected imaging agent is an indicator of the location and amount of the biomarker SAS1R.

In one embodiment, the cancer is selected from the group consisting of lung cancer, MMMT, bladder cancer, ovarian cancer, uterine cancer, endometrial cancer, breast cancer, head and neck cancer, liver cancer, pancreatic cancer, esophageal cancer, stomach cancer, cervical cancer, prostate cancer, adrenal cancer, lymphoma, leukemia, salivary gland cancer, bone cancer, brain cancer, cerebellar cancer, colon cancer, rectal cancer, colorectal cancer, oronasopharyngeal cancer, NPC, kidney cancer, skin cancer, melanoma, basal cell carcinoma, hard palate carcinoma, squamous cell carcinoma of the tongue, meningioma, pleomorphic adenoma, astrocytoma, chondrosarcoma, cortical adenoma, hepatocellular carcinoma, pancreatic cancer, squamous cell carcinoma, and adenocarcinoma.

In one aspect, the cancer is a metastatic cancer.

The invention is also useful for comparing the levels of SAS1R being imaged to help determine whether a cancer is benign or malignant, based on the level of imaging agent detected (a measure of the amount of SAS1R).

The invention is also useful for determining the stage of carcinogenesis of a cancer and monitoring its progression from early to late stage cancer. This method is useful for determining the type and amount of therapy to use.

Optionally, a therapeutic agent can be attached or can be included in a pharmaceutical composition comprising the imaging complex.

Clinically relevant PET/SPECT tracers as used herein enable the detection of small tumors and metastases.

In one aspect, the imaging agent or detectable moiety includes, but is not limited to, a radionuclide, a radiological contrast agent, a paramagnetic ion, a metal, a biological tag, a fluorescent label, a chemiluminescent label, an ultrasound contrast agent and a photoactive agent.

The antibody, or a homolog or fragment thereof, imaging complexes of the invention encompass detecting, diagnosing, and localizing cancers other than the ones disclosed herein, as long as the cancer is a SAS1R positive cancer. The present invention further provides for quantifying levels of SAS1R, and thus encompasses the ability to distinguish normal, benign, and malignant tissue.

The invention includes detection of SAS1R microRNAs in blood samples as biomarkers. miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules.

The present invention further provides kits comprising at least one antibody ligand complex of the invention, an instructional material, and optionally includes at least one imaging agent and optionally at least one therapeutic agent.

The present invention provides multiple techniques for measuring SAS1R mRNA and protein expression and levels, including but not limited to, PCR, northern blots, western blots, immunohistochemistry, and immunofluorescence.

Treating Cancer

The present invention provides compositions and methods for treating cancers expressing SAS1R. In one aspect, the expressed SAS1R is a protein. In one aspect, the present invention encompasses the use of an antibody capable of binding specifically to SAS1R on the surface of a cancer cell. In one aspect, the antibody can bind to one of the epitopes described herein. In one aspect, the antibody can binding to one of the sequences and fragments disclosed herein. Said antibody can be, for example, a single chain antibody, a monoclonal antibody, a bi-specific antibody, a chimeric antibody, a synthetic antibody, a polyclonal antibody, a humanized antibody, a human antibody, or active fragments or homologs thereof. In one aspect, a therapeutic agent is coupled to the antibody.

In one embodiment, the present invention provides antibodies useful for diagnosing and treating cancer, wherein said antibodies bind to SAS1R. In one embodiment, the present invention provides antibodies useful for diagnosing and treating cancer, wherein said antibodies bind to an epitope of SAS1R. In one aspect, the present invention provides pharmaceutical compositions comprising antibodies of the invention.

Many therapeutic agents are available that can be conjugated to an antibody directed against SAS1R and used in combination with the antibody. For example, molecules that can be attached to SAS1R include, but are not limited to, pro-drugs, drugs, toxins, protein toxins, liposomes, filled liposomes, radioactive isotopes, and enzymes. The use of antibody-enzyme conjugates directed at tumor-associated antigens to achieve site-specific activation of prodrugs to potent cytotoxic species, termed “antibody-directed enzyme prodrug therapy” (ADEPT). In one aspect, the antibody directed against SAS1R is useful for treating cancer by antibody-mediated complement-dependent cell death.

Because of the stage specific expression of SAS1R, if targeted for therapy in a cancer patient, the reserve of oocytes contained in primordial and primary follicles in the ovary would be preserved. That is because, not only is SAS1R specific for the oocyte, but it is expressed in oocyte in a precise temporal and spatial manner. For example, SAS1R expression is specific for particular stages of oocyte development during follicular maturation (data not shown). SAS1R proteins appear in ovaries only in those oocytes that have reached the secondary follicle stage of follicular maturation. SAS1R then persists only in the oocytes within subsequent stages of pre-antral and antral follicles. The first stage at which SAS1R appears, the secondary follicle, is defined as those oocytes that are surrounded by two or more layers of granulose cells. This finding indicates that using SAS1R as a drug target, vaccine, or surface target for gene or drug delivery would permit the selective attack on only the pool of maturing oocytes and not oocytes contained within primordial and primary follicles. In other words, this is the definitive demonstration that targeting SAS1R would spare the ovarian reserve of immature oocytes.

Because among adult tissues SAS1R is expressed only in oocytes and as disclosed herein in tumors, the methods of the present invention which would target SAS1R means that its use as a drug target or as a vaccine would allow for selective targeting of the cells and tissues that express SAS1R.

Additionally, it is known that SAS1R is an active enzyme, and is thus a drugable target for cancer therapy.

A cancer drug target, such as the one disclosed herein, fulfills essential criteria for targeted cancer therapy, as well as for sparing normal cells that do not express the target.

The present invention provides compositions and methods useful for inhibiting the interaction of SAS1R with other proteins. The present application further provides for the use of antibodies directed against SAS1R to detect and treat cancer. In one aspect, the type of antibody includes, but is not limited to, a polyclonal antibody, a monoclonal antibody, a chimeric antibody, and a synthetic antibody. In one aspect, the antibody is a monoclonal antibody. The invention further provides hybridomas comprising monoclonal antibodies of the invention. The invention further provides sequences and fragments of antibodies of the invention.

Inhibitors of SAS1R include those which inhibit its interaction or binding with a sperm protein such as SLLP1 or any other protein, its activity as a protease, or inhibit its regulation of downstream activities included in SAS1R signal transduction pathways, including its role in cancer cells. In one aspect, the inhibitor is SAS1R, or a fragment or homolog of SAS1R which binds with SLLP1. In one aspect, the SAS1R fragment is an N-terminus portion of the protein. In one aspect, the N-terminus comprises about the amino terminal 121 amino acid residues of mature SAS1R. In another aspect, the SAS1R fragment which binds with SLLP1 and inhibits SLLP1 interaction with an egg is a C-terminus portion of SAS1R. In one aspect, the C-terminus of the SAS comprises about the carboxy terminal 210 amino acid residues of SAS1R. One of ordinary skill in the art will appreciate that any kind of compound that inhibits SAS1R levels, function, or activity as described herein, or those that are yet unknown, are encompassed by the present invention.

An inhibitor of SAS1R can be any type of molecule that inhibits, for examples, SAS1R function, activity, expression, and protein levels. In one aspect, SAS1R is inhibited in an N-terminus portion of the protein. In one aspect, the N-terminus comprises about the amino terminal 121 amino acid residues of mature SAS1R. In another aspect, the SAS1R is inhibited in a C-terminus portion of the protein. In one aspect, the C-terminus of the protein comprises about the carboxy terminal 210 amino acid residues of SAS1R. In one aspect, the inhibitor is an antibody directed against SAS1R. In another aspect, the inhibitor is a drug or other compound. In one aspect, the inhibitor inhibits the protease activity of SAS1R. In another aspect, the inhibitor inhibits the interaction of SAS1R with SLLP1 or any other protein. In one aspect, the interaction is binding.

The present inventors have surprisingly found that suitable antigens for immunotherapeutic strategies include the protein SAS1R. The present application discloses immunogenic compositions comprising an immunogen that is derived from eggs in normal cells, which as disclosed herein is also expressed in, for example, lung, uterine and ovarian cancers. That antigen is the SAS1R protein, as well as antigenic fragments and homologs thereof. The present application demonstrates that cells expressing SAS1R can be killed using such strategies.

The present invention provides compositions and methods useful for detecting and diagnosing cancer, e.g., kidney cancer, and for treating cancer, such as kidney cancer.

In one aspect, SAS1R, or fragments or homologs thereof which maintain the immunogenic activity of full length SAS1R, can be administered to a subject to elicit an immune response against SAS1R. In one aspect, the administration of SAS1R and fragments and homologs thereof which elicit an immune response is useful as a vaccine. In one aspect, it is a vaccine against cancer. In one aspect, the cancer is lung, uterine cancer or ovarian cancer. In one aspect, the cancer is a malignant mixed mullerian tumor. In one aspect, the ovarian cancer is a serous ovarian cancer.

SAS1R isoforms are found on the cell surface in oocytes and in transfected cells, as well as on cancer cells as described herein. Therefore, in cancer cells expressing SAS1R on the surface, SAS1R is a tumor selective surface target. In one aspect, cancer cells can be targeted with an antibody directed against SAS1R. In one aspect, the antibody is a humanized antibody.

In one aspect, the antibody has a toxin or drug linked to it for delivery to a cancer cell.
In one embodiment, the compositions and methods of the invention are useful in mammals. In one aspect, the mammal is a human.

The SAS1R enzyme shows stage specific expression in secondary and subsequent follicular oocytes, including preantral and antral follicles (data not shown), rendering it a suitable target for cancer therapy or as a vaccine that will spare naked, primordial, and primary oocytes, and preserve the ovarian reserve of germ cells. Therefore, the invention further encompasses the compositions and methods for identifying compounds that inhibit SAS1R.

The present invention further provides methods for treating cancer. In one aspect, the invention provides compositions and methods for treating cancer cells expressing SAS1R. In one aspect, SAS1R is a cell surface protein. The methods for treating cancer cells expressing SAS1R include those described herein and other known methods which can target SAS1R and its activity.

The linkage of a diagnostic biomarker to a specific therapy will result in the “intelligent” treatment of cancer by identifying subjects whose disease will respond to a specific treatment. This is often referred to as “individualized therapy” or “personalized medicine”. For example, an ovarian or uterine cancer biomarker would greatly reduce the costs associated with drug development by enabling the selection of a more homogeneous patient population for smaller, more cost-effective clinical trials. A useful biomarker can also accelerate drug development by facilitating decisions regarding which agents to pursue in the early stages of clinical development. By emphasizing targets that can be both a successful diagnostic or screening test and a therapeutic drug or vaccine target, particular advances may be possible.

The present invention provides a means to phenotype a subject's tumor to identify the SAS1R signature. This subject will likely benefit from a SAS targeted therapy. Such therapy can be a first or “front line” therapy, used before more traditional chemotherapeutic agents which induce weakness, hair loss, diarrhea and anemia, due to their non selective mechanisms of action.

The present invention further provides for altering the actin distribution in a cancer cell and altering the appearance of the cells, comprising contacting said cell with an antibody directed against SAS1R, wherein said antibody binds with SAS1R and the actin distribution of the cell is altered and the appearance of the cell is altered.

The present invention further provides compositions and methods utilizing SAS1R, and fragments and homologs thereof, to elicit an immune response against SAS1R. In one aspect, administration of SAS1R or antigenic fragments or homologs thereof results in inhibiting SAS1R. In one aspect, such an immunogenic response and resulting inhibition of SAS1R function.

Many studies have demonstrated that at the time of diagnosis, a cancer patient already has circulating cancer cells within his or her blood stream. It is not uncommon for 5 or 10 cancer cells to be found in each milliliter of blood. This means that it is not uncommon for thousands of cancer cells to be present in virtually every organ, as potential metastases, at the time of diagnosis. The survival of even a tiny fraction of these cells will result in the development of a metastasis, and reduce the survival of the patient.

The present invention encompasses techniques demonstrated herein to detect SAS1R, and one of ordinary skill in the art will appreciate that other techniques can be used as well. Detecting and measuring protein can be done in many ways. For Example, useful methods include, for example, performing LC-MS/MS analyses, which can be performed on a ThermoFinnigan LCQ Deca ion trap MS instrument equipped with a ThermoFinnigan Surveyor HPLC pump and microelectrospray source and operated with ThermoFinnigan Xcalibur version 1.2 system control and data analysis software. Analysis of samples can be performed with an acetonitrile gradient and a Monitor C18 (Column Engineering) packed tip with 100 μm ID, 360 μm OD, and 5-15 μm tip opening. The flow from the HPLC pump can be split to achieve 500 nL to 1 μl flow rate from the packed tip. Two gradients can be used, “fast” and “normal”, depending on the complexity of the sample being analyzed.

A protein can be subjected to Tandem Mass Spectroscopic Analysis and peptide sequences obtained.

In one embodiment, the invention provides a pharmaceutical composition comprising a pharmaceutically-acceptable carrier and SAS1R, or a homolog, fragment or derivative thereof, wherein said protein is capable of inducing an immune response in a subject. In one aspect, the method is useful as a vaccine or as a treatment. In one aspect, the invention provides a pharmaceutical composition, wherein said egg protein or peptide comprises an amino acid sequence selected from the group consisting of SEQ ID NOs:6, 8, 10, 19, 20, 21, and 23, and fragments and homologs thereof.

In one embodiment, at least one isolated nucleic acid comprising a nucleic acid sequence encoding an egg protein is administered. In one aspect, the egg protein comprises a sequence selected from the group consisting of SEQ ID NOs: 6, 8, 10, 19, 20, 21, and 23, and fragments and homologs thereof.

An administered protein or a protein expressed by an administered isolated nucleic acid comprising a sequence encoding the protein can act to inhibit SLLP1 and SAS1R interaction or binding.

The present invention also provides for administering at least one SLLP1 protein or biologically active homologs and fragments thereof capable of binding with or interacting with SAS1R to detect SAS1R. In one aspect, the SLLP1 protein is labeled.

It will be appreciated, of course, that the proteins or peptides of the invention may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

### Examples

Sas1B Internalizes in Uterine Cancer Cells In Vitro and Co-Localizes with Proteins in the Endocytic Pathway.

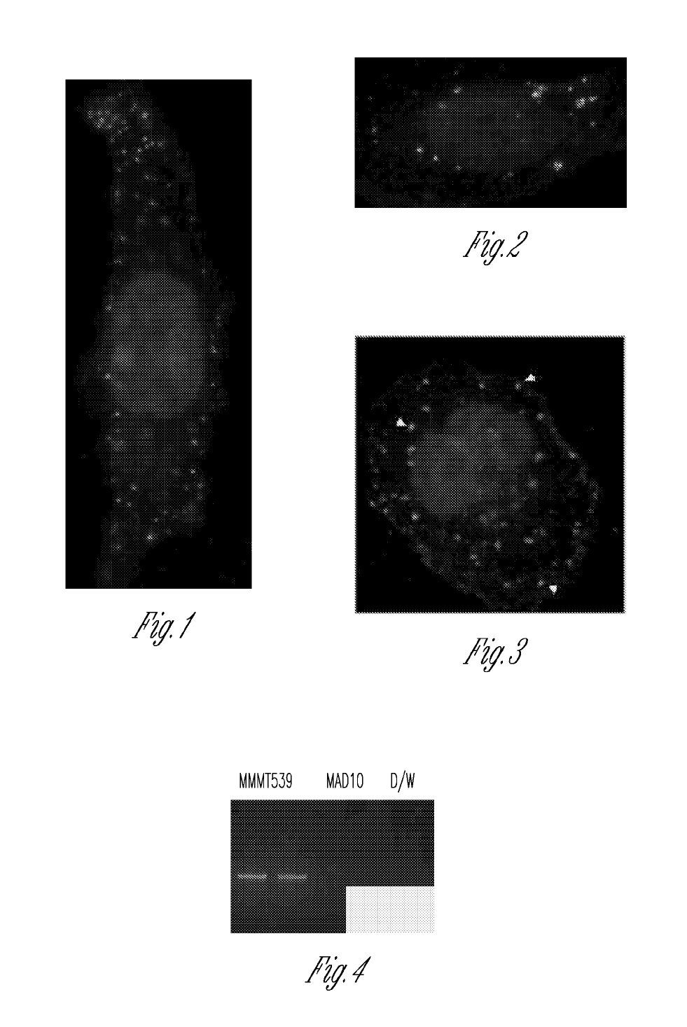

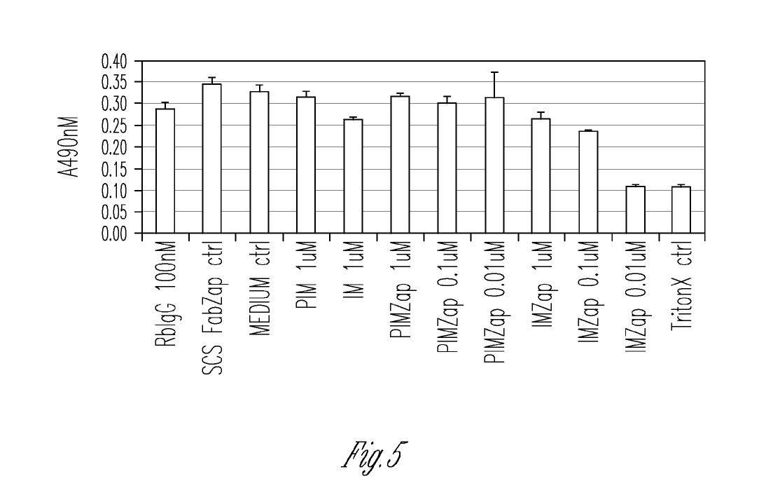

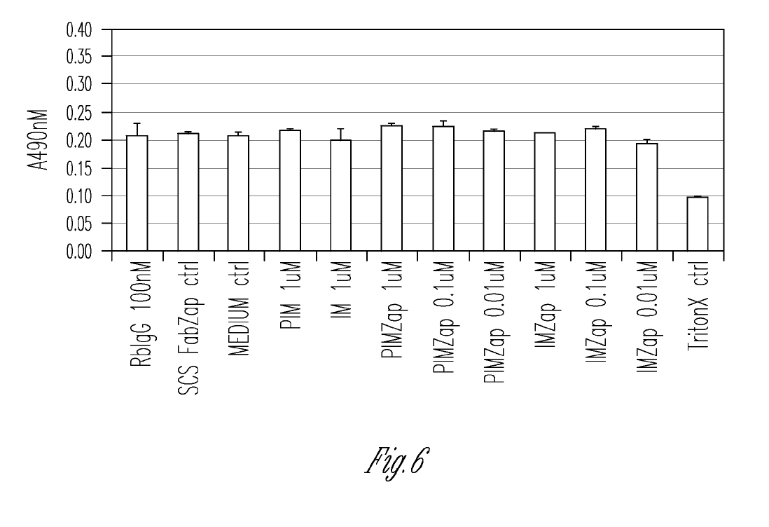

FIG. 1 shows MMMT 538 cells that were incubated with antibody to SAS1B at 4° C., warmed to 37° C. for 15 min, and then stained for the location of the SAS1B antibody. Small SAS1B antibody positive vesicles were observed at the cell periphery just beneath the cell membrane. FIG. 2 shows an MMMT 539 cell after similar treatment, but after 60 minutes of incubation at 37° C. SAS1B antibody positive vesicles were found to be larger and more deeply internalized in the cytoplasm. These results indicate that SAS1B is internalized in MMMT cells. SAS1B positive vesicles co-localized with components of the early and late arms of the endocytic pathway. FIG. 3, for example, shows yellow vesicles (arrowheads) where green SAS1B positive vesicles co-localized with red vesicles staining for EEA1, a marker of the early arm of the endocytic pathway. Similar results have been noted with LAMP1, a marker of the late arm of the endocytic pathway.

Cell Lines with and without SAS1B.

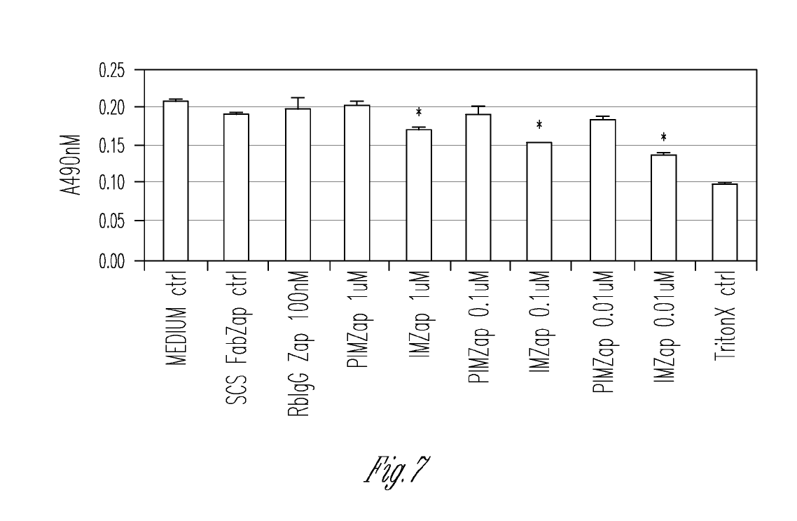

FIG. 4 shows the 310 bp c-term SAS1B amplimer in MMMT 539 cells, but not in the virus transformed uterine stromal cell line MAD10, demonstrating that MMMT 539 cells express SAS1B, whereas MAD10 cells do not.

Immunotoxins Arrest Growth of SAS1B Bearing Cells, but not Cells Lacking SAS1B.

As SAS1B is found among normal tissues only in the oocyte, when SAS1B is expressed in tumors, its presence offers an opportunity to achieve a mechanism of chemotherapeutic drug action that is tumor selective. Many current cancer therapeutics, such as Cisplatin or Carbotaxol, target proteins involved in central, generalized pathways that are present in a wide variety of organ systems. These include proteins involved in cell replication; signal transduction, mitosis and cell migration. As a result, many contemporary chemotherapeutics induce nausea, hair-loss, diarrhea, skin lesions and deplete germ cells making these drugs difficult for many individuals to endure and requiring cryopreservation of gametes to overcome infertility. The approach discussed herein provides a selective mechanism of drug action that targets only the tumor and an expendable population of mature oocytes. The strategy for targeting SAS1B is designed to spare oocytes that constitute the ovarian reserve within primordial follicles and prevent the side-effect of infertility that is common in contemporary chemotherapeutics. The absence of SAS1B in the oocyte reserve, the restriction of SAS1B to growing oocytes among normal tissues, and SAS1B's cell surface expression in tumors affords a novel opportunity to create a unique tumor-selective biologic drug. This is novel approach involves the use of SAS1B as a chemotherapeutic drug target.

This can be carried using an antibody-drug conjugate. For example, immunotoxin drugs combined with both polyclonal and monoclonal immunoreagents can be used. Murine monoclonal antibodies may be humanized by grafting onto a human immunoglobulin scaffold to create a non-immunogenic recombinant humanized antibody. This antibody can then be used as a “naked” human antibody therapeutic, or conjugated with toxins, peptides, small molecule drugs or radionuclides.

Fab Saporin Cytoxicity Assay Protocol

Day 1

Materials: MMMT539 uterine cell lines; TrypLE Select for trypsinization; 96 well tissue culture plates; Primary antibodies: rabbit (Rb) sera to hSAS1B (Immune and Pre-Immune); Advanced Targeting Systems Rabbit Fab-ZAP; RPMI media with heated FBS for M539 cells.

SRB (Sulforhodamine B) processing reagents: DPBS (without calcium or magnesium); 50% Trichloroacetic acid (TCA) (stored @ 4° C.); 0.05% Sulforhodamine B (SRB) (Sigma cat #S-1402) made in 1% Acetic acid; and 10 mM Tris (non buffered).

All steps (until endpoint processing) were carried out under sterile/aseptic conditions.

Preparation of Cells: Trypsinize cells with TrypLE to help maintain antigen integrity; add 2 ml TrypsinLE and swirl contents, aspirate and replace with fresh 2-3 ml trypsin till cells float; gently tap sides to loosen all cells; add 5 ml fresh warm medium to stop action of trypsin and squirt cells with same medium several times; collect in 15-50 ml conical tubes; centrifuge at 3000 rpm for 5 mins.; throw away supernatant and to the cell pellet add 1 ml of fresh medium; conduct a total cell count.

Cell count and re-plating: Count cells on hemocytometer or any cell counting chambers; adjust cell count to have 4000 cells/200 ul medium as final cell count (make this number for number of wells that will be utilized; in triplicate); plate cells in 200 ul medium/well in 96 well plates; and incubate overnight at 37° C. in 5% CO2. Note: Densities vary depending on cell size and doubling time. After 5 days control wells should not be confluent and SRB processing should have an OD at 540 of less than 2.0 to be in linear range.

Fab Saporin Cytoxicity Assay Protocol

Day 2

Primary Antibody dilutions (PAD): 1. Rabbit IgG Immune [IM] (21-10-11) 4 mg/ml. PAD-IM; 2. Rabbit IgG Pre-Immune [PIM] (21-10-11) 1.2 mg/ml. PAD-PIM.

Rb IgG ZAP: This is commercially available Rb IgG which is direct tagged with Saporin and is used as control. 1 ul of it+9 ul of RPMI. From this, 3 ul was mixed with 60 ul of media and 20 ul was added per well (10 nM).

Tx100 positive control: 1 ul of Tx100 (SIGMA)+99 ul media. Mix well. Add 20 ul per well.

Secondary conjugate solution (SCS): 45 ng/10 ul or 90 ng/20 ul is used per well for the assay. For secondary conjugate, concentration of this conjugate as provided by manufacturers is 2.3 mg/ml. 1 ul of this conjugate is needed in 499 ul of medium. Vortex for 5 secs.

Conjugation: For PIM and IM, each tube has a 1:10 dilution of the previous concentration of the primary antibody.

PIM1: 10 ul of PAD-PIM+63 ul of SCS. Mix well for 5 secs. (1 uM)

PIM2: 7.5 ul of PIM1 sol+67.5 ul of SCS. Mix well for 5 secs. (0.1 uM)

PIM3: 7.5 ul of PIM2 sol+67.5 ul of SCS. Mix well for 5 secs. (0.01 uM)

PIM4: 7.5 ul of PIM3 sol+67.5 ul of SCS. Mix well for 5 secs. (1 nM)

PIM5: 7.5 ul of PIM4 sol+67.5 ul of SCS. Mix well for 5 secs. (0.1 nM)

PIM6: 7.5 ul of PIM5 sol+67.5 ul of SCS. Mix well for 5 secs. (0.01 nM)

PIM7: 7.5 ul of PIM6 sol+67.5 ul of SCS. Mix well for 5 secs. (1 pM)

PIM8: 7.5 ul of PIM7 sol+67.5 ul of SCS. Mix well for 5 secs. (0.1 pM)

IM1: 3 ul of PAD-IM+72 ul of SCS. Mix well for 5 secs.

IM2: 7.5 ul of IM1 sol+67.5 ul of SCS. Mix well for 5 secs.

IM3: 7.5 ul of IM2 sol+67.5 ul of SCS. Mix well for 5 secs.

IM4: 7.5 ul of IM3 sol+67.5 ul of SCS. Mix well for 5 secs.

IM5: 7.5 ul of IM4 sol+67.5 ul of SCS. Mix well for 5 secs.

IM6: 7.5 ul of IM5 sol+67.5 ul of SCS. Mix well for 5 secs.

IM7: 7.5 ul of IM6 sol+67.5 ul of SCS. Mix well for 5 secs.

IM8: 7.5 ul of IM7 sol+67.5 ul of SCS. Mix well for 5 secs.

All of these dilutions are allowed to incubate in hood for 45 minutes at room temperature.

Fab Saporin Cytoxicity Assay Protocol

Day 3

Sulforhodamine B Staining:

1. Once desired time point was achieved, media was removed and saved; 2. 200 ul of chilled DPBS was added per well; 3. to this was added 50 ul of chilled 50% TCA per well; 4. it was then allowed to incubate at 4C for 1 hr; 5. wash with 300 ul TAP water per well about 4-5 times and pat dry; 6. air dry for 1 hr or till done (plates can be stored at this step indefinitely); 7. add 100 ul of a 1× working Sulforhodamine reagent which was made in 1% acetic acid and incubate at RT for 30 mins.; 8. wash with 1% acetic acid 300 ul per well 5-6 times or till pink color from washes disappear; 9. pat dry and air dry completely (this can be stored at this stage till the next step); 10. solubilize with 200 ul per well non-buffered Tris base reagent and mix on shaker or with pipette tip completely. Shake for 5-10 mins; 11. read at 490 nm and plot on graph.

An indirect saporin assay employing the sulforhodamine B screening method to quantify cell density and growth arrest was performed with MMMT 539 and MAD 10 cells to test the hypothesis that the SAS1B pathway could be used for intracellular delivery of a toxic cargo (FIG. 5). The assay employed IgGs that were purified from rabbit polyclonal antiserum to recombinant human SAS1B as the primary antibody, and a secondary anti-rabbit Fab-Zap reagent containing saporin from Advanced Targeting Systems. Cell killing was observed only with anti-SAS1B primary IgG antibodies (red bars labeled IMZAP) and not with any of the negative controls, including pre-immune IgGs used at identical concentrations as immune IgGs (yellow bars labeled PIZAP), purified nonspecific rabbit IgG labeled with saporin (RbIgG), or the secondary Fab-ZAP saporin reagent alone (SCS FABZAP ctl). Levels of cell killing were concentration depended with the assay exhibiting a prozone (Hook) effect. At high primary antibody concentration the Fab-ZAP toxin complexed with the primary antibody in solution, rather than to primary antibody bound at the cell surface. However, when the stoichiometry of primary antibody and secondary ZAP reagent was optimized to allow the Fab-ZAP immune complexes to reach the cell surface, complete growth arrest was observed. This arrest was at the same level as that seen with tumor cells that had been lysed at the beginning of the treatment phase with non-ionic detergents (green bar, Triton X positive control).

Complete cell growth arrest was observed at 10 nM anti-SAS1B IgGs and 5.14 nM Fab-ZAP saporin. No cell growth arrest effects were observed with any of the reagents in MAD10 cells which do not express SAS1B. These studies were replicated 9 times with identical results. The results were confirmed by morphological observation of cells which demonstrated that only anti-SAS1B treated cells had rounded up and became psychotic. These studies demonstrate that SAS1B is internalized into the endocytic pathway and this pathway may be used for intracellular delivery of a toxic cargo.

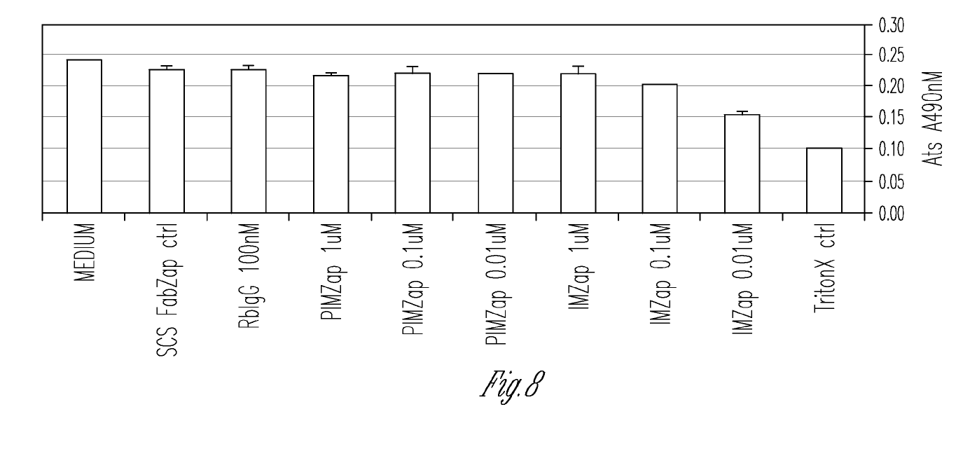

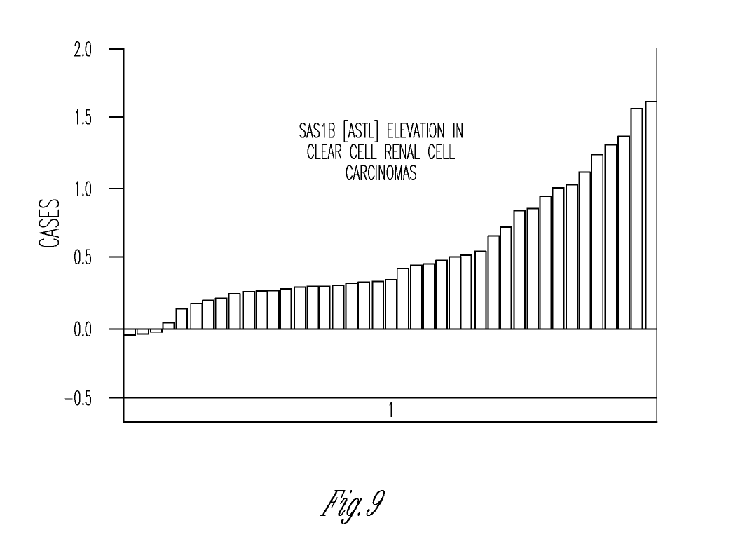

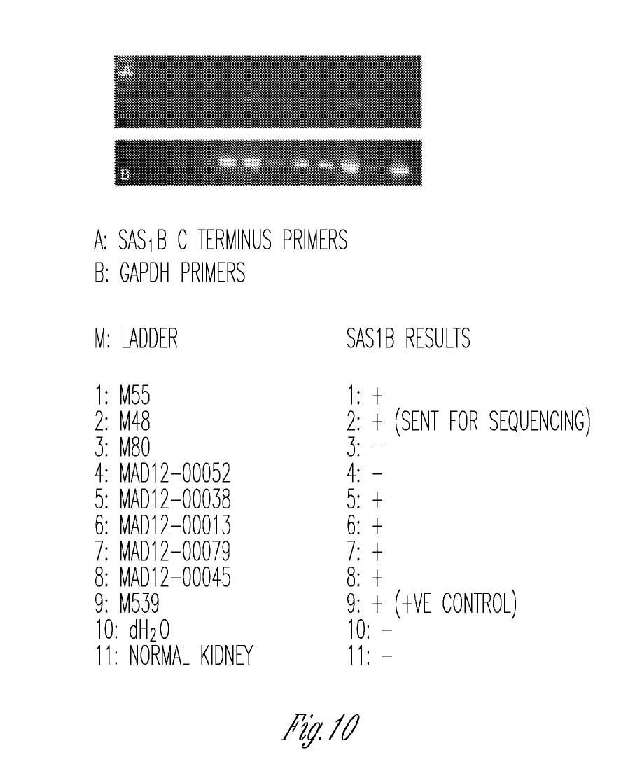

Thus, saporin piggy-back on secondary antibody (Gt anti-rabbit) was effective in targeting saporin into the cell cytoplasm of M539 uterine cance cells by endocytosis. 0.01 uM concentration of IM IgG's was found to be effective. No effect was seen with control uterine cells (MAD10) (see FIGS. 6-8). Treated M539 cells show a loss of contact with other cells, loss of architecture and cell shape change, and presence of blebs/pores/vacuoles in cytoplasm of cells. M539 cell culture supernatant was checked for LDH activity and IM concentration at 0.01 uM showed significant increase in LDH levels.

### SAS1B is Expressed in Renal Tumors

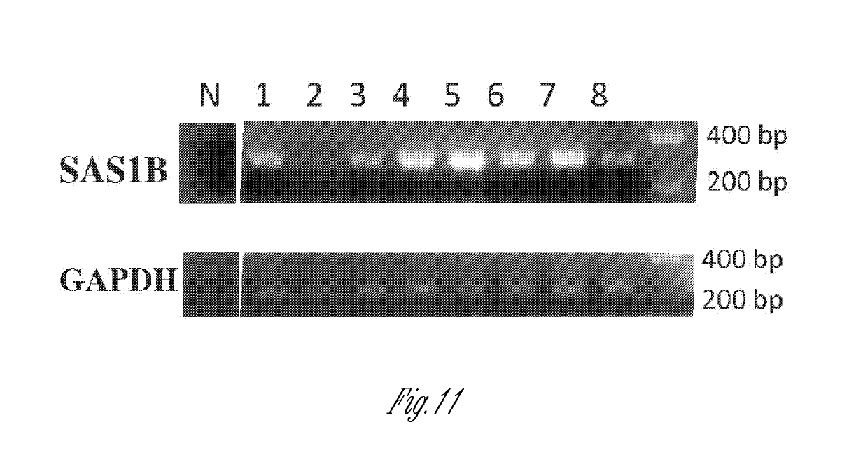

Oncomine (a cancer microarray database and web-based data-mining platform aimed at facilitating discovery from genome-wide expression analyses) interrogations demonstrate that SAS1B (ASTL) is elevated in clear cell renal cell carcinomas (FIG. 9). Thus, SAS1B can be used as a diagnostic marker for screening large populations of patients with renal tumors. There are approximately 65,000 new renal tumor cases each year and about 14,000 deaths each year. 90% of the cases are renal carcinoma, of which about 70% of the cases are clear cell carcinoma and about 10% of the cases are papillary carcinoma. SAS1B is membrane associate in renal tumors. cDNA from clear cell renal tumor from patients were used in the PCR assay using c-term SAS1B primers (panel A) and to ensure integrity of RNA, GAPDH primers were used as seen in panel B. M539 uterine cancer cell lines were used as a positive control as seen in lane 9. Normal kidney cDNA served as a tissue control in 11 and does not express SAS1B (FIG. 10). Using the gene sequence in the Blastn program resulted in 99% identity to ASTL astacin-like metallo-endopeptidase (M12 family) (Gene ID: 431705).

### Sas1B is Expressed in Head and Neck Squamous Cell Carcinoma

Head and neck squamouos cell carcinoma (HNSCC) is the fifth leading cancer diagnosis and the eighth leading cause of cancer death worldwide. Great than 90% of head and neck cancers are squamous cell carcinomas. This type of cancer develops in squamous cells lining the moist surfaces of the upper aerodigestive tract.

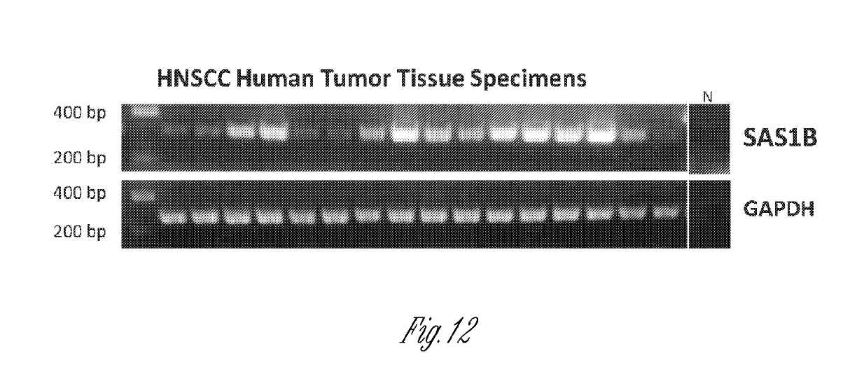

SAS1B mRNA and protein are observed in 100% of HNSCC cells lines (FIG. 11). Briefly, total RNA was extracted (Qiagen kit) from eight HNSCC cell lines and converted to cDNA. PCR amplification was performed using C-terminal hSAS1B primers. 8/8 HNSCC lines were positive for SAS1B transcript. Tumors numbered 1-8; N=negative template control (water). hGAPDH used as a loading control.

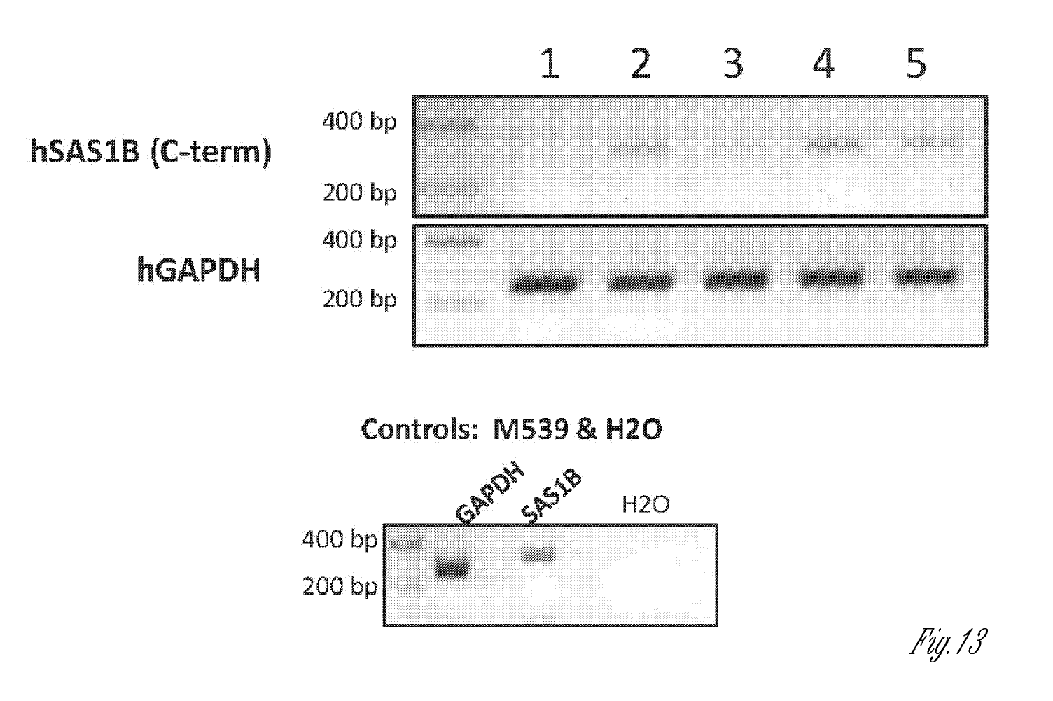

100% of HNSCC human tumors investigated were positive for SAS1B (FIG. 12). FIG. 12 representative of PCR results for SAS1B mRNA amplification from HNSCC human tumor specimens. Total RNA was extracted from tumor tissue (Qiagen) and reverse transcribed to cDNA. PCR amplification was performed using C-terminal hSAS1B primers. Lane order left to right: marker, HNSCC tumor specimens, N=negative template, H₂O control. A total of 24 tumor specimens were tested; 24/24 were positive for SAS mRNA, 16 of which are shown here. hGAPDH used as a loading control.

### SAS1B is Expressed in Pancreatic Cancer

Pancreatic cancer is the fourth leading cause of cancer deaths in the United States. The location of pancreatic cancer, retroperitoneum—behind the stomach, complicates diagnosis and treatment. 65% of pancreatic cancers are located in the head of the pancreas, while 15% are located in the body, 10% in the tail and 10% are multifocal. Greater than 90% of pancreatic cancers are adenocarcinomas of pancreatic origin. Generally, these cancers have a disorganized ductal distribution, anisonucleosis (>4:1—variation in size of nuclei) and grandular mitotic figures are present.

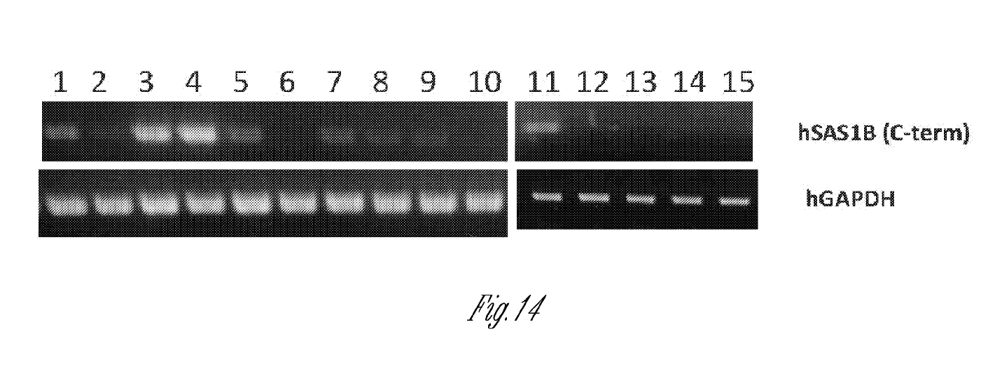

SAS1B transcripts were identified in pancreatic cancer cell lines (FIG. 13). Briefly, total RNA was extracted (Qiagen kit) from five pancreatic cancer cell lines and converted to cDNA. PCR amplification was performed using C-terminal hSAS1B primers. 4/5 pancreatic cancer lines were positive for SAS transcript. Control reactions shown: SAS1B expression in M539 cells (malignant mixed Mullerian tumor) as positive control and GAPDH loading control for M539; no template (water) control. hGAPDH used as a loading control.

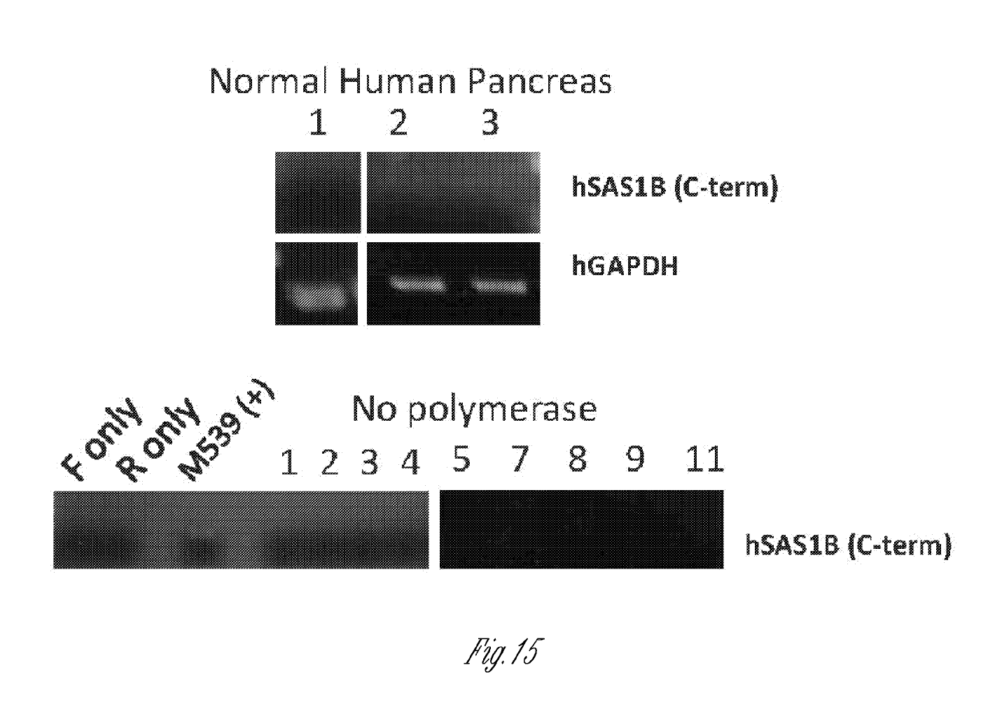

SAS1B in was identified in pancreatic tumors of both males and females (FIG. 14). Total RNA was extracted (Qiagen kit) from 15 human pancreatic tissues xenografted orthotopically into mice and then passaged. Specimens examined were of approximately generation 5 xenografts. RNA was reverse transcribed to cDNA. PCR amplification was performed using C-terminal hSAS1B primers. 9/15 pancreatic cancer lines were positive for SAS1B transcript. hGAPDH used as a loading control.

FIG. 15 provides pancreatic PCR controls. Total RNA was extracted (Qiagen kit) from 3 human normal pancreas tissues. RNA was reverse transcribed to cDNA. Top: PCR amplification was performed using C-terminal hSAS1B primers in normal pancreas. All three normal pancreas were negative for SAS1B transcript. hGAPDH used as a loading control. Bottom: PCR amplification was performed using hSAS1B C-terminal primers. Single primer control reactions for pancreatic tumor. M539 (MMMT line) used as positive control.

### BIBLIOGRAPHY

Mandal A, et al. (2003) Biol Reprod 68, 1525-1537.
Herrero M B, et al. (2005) Dev Biol 284, 126-142.
Evans J P, Schultz R M, Kopf G S (1995) J Cell Sci 108, 3267-3278.
Almeida E A, et al. (1995) Cell 81, 1095-1104.
Yuan R, Primakoff P, Myles, D G (1997) J Cell Biol 137, 105-112.
Evans J P, Schultz R M, Kopf G S (1997) Dev Biol 187, 94-106.
Cho C, et al. (1998) Science 281, 1857-1859.
Shamsadin R, et al. (1999) Biol Reprod 61, 1445-1451.
Nishimura H, Cho C, Branciforte D R, Myles D G, Primakoff P (2001) Dev Biol 233, 204-213.
Coonrod S A, et al. (1999) Dev Biol 207, 334-349.
Miyado K, et al. (2000) Science 287, 321-324.
Alfieri J A, et al. (2003) J Cell Sci 116, 2149-2155.
Kaji K, et al. (2000) Nat Genet 24, 279-282.
Le Naour F, Rubinstein E, Jasmin C, Prenant M, Boucheix C (2000) Science 287, 319-321.
Rubinstein E, et al. (2006) Dev Biol 290, 351-358.
Cohen D J, Ellerman D A, Cuasnicu P S (2000) Biol Reprod 63, 462-468.
Da Ros V G, et al. (2008) Dev Biol 320, 12-18.
Inoue N, et al. (2005) Nature 434, 234-238.
Luban J, Goff S P (1995) Curr Opin Biotechnol 6, 59-64.
Li X, McDermott B, Yuan B, Goff S P (1996) J Virol 70, 1266-1270.
Miyado K, et al. (2008) Proc Natl Acad Sci USA 105, 12921-12926.
Weigmann A, Corbeil D, Hellwig A, Huttner W B (1997) Proc Natl Acad Sci USA 94, 12425-12430.
Evans J P, et al. (2000) Biol Reprod 62, 76-84.
Goldman S, Shalev E (2003) Eur J Obstet Gynecol Reprod Biol 111, 109-121.
Robinson L L, Sznajder N A, Riley S C, Anderson R A (2001) Mol Hum Reprod 7, 641-648.
Elia L, Marsh L (1998) J Cell Biol 142, 1473-1485.
Roe J L, Farach H A, Strittmatter W J, Lennarz W J (1988) J Cell Biol 107, 539-544.
Correa L M, Cho C, Myles D G, Primakoff P (2000) Dev Biol 225, 124-134.
Hiroi J, et al. (2004) Dev Genes Evol 214, 176-184.
Quesada V, Sanchez L M, Alvarez J, Lopez-Otin, C (2004) J Biol Chem 279, 26627-26634.
Bond, J., et al., Protein Science (1995), 4: 1247-1261.
Möhrlen, F., et al., 2008, 8:2, 223-231.
Mandal, A., et al., Biology of Reproduction 78: 69.72 (2008).
Rosmann, S., et al., The Journal of Biological Chemistry, Vol. 277, No. 43, Issue of Oct. 25, 2002, pp. 40650-40658.
Sterchi, E., et al., Molecular Aspects of Medicine, 29 (2008) 309-328.
Huguenin, M., et al., PLos One, May 2008, Vol. 3, Issue 5, e2153, p. 1-11.
Barbara A. Weir, et al 2007 Nature 450, 893-898.
Martin L. Sos, et al 2009 J. Clin. Investigation 119, 6 1727-1740.
Rhodes D R. Yu J. Shanker K. Deshpande N. Varambally R. Ghosh D.
Barrette T. Pandey A. Chinnaiyan A M. 2004 (Jun. 22, 2004).
Rhodes D R, Yu J, Shanker K, Deshpande N, Varambally R, Ghosh D,
Barrette T, Pandey A, Chinnaiyan A M. Neoplasia. 2004 January-February; 6(1):1-6.
Vanicha Vichai & Kanyawim Kirtikara. Nature Protocols 1, -1112-1116 (2006).

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

What we claim is:

1. An immunotoxin molecule comprising an antibody, or a binding fragment thereof, specific for SAS1R antigen conjugated to a cytotoxic agent.

2. The immunotoxin molecule of claim 1, wherein the cytotoxic agent is a Type I ribosome inactivating protein.

3. The immunotoxin of molecule of claim 2, wherein the Type I ribosome inactivating protein is saporin.

4. The immunotoxin molecule of claim 1, wherein the antibody is monoclonal, polyclonal, chimeric, human, or humanized.

5. The immunotoxin molecule of claim 1, wherein the antibody binding fragment is F(ab′)₂, F(ab)₂, Fab′, or Fab.

6. A composition comprising the immunotoxin according to claim 1 and a physiologically acceptable carrier.

7. A method of killing, inhibiting or sloughing off of cancer cells comprising administering to a subject in need thereof at least one immunotoxin molecule of claim 1.

8. The method of claim 7, wherein the cancer is selected from the group consisting of carcinoma (e.g., head and neck squamous cell carcinoma), sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, neuroblastoma, melanoma, breast cancer, prostate cancer, pancreatic cancer, kidney cancer, bladder and endometrioid carcinoma.

9. A method to treat cancer, comprising administering a therapeutically effective dose of an immunotoxin conjugate to a subject in need thereof, wherein said immunotoxin conjugate comprises an antibody, or a binding fragment thereof, conjugated to a cytotoxic agent, wherein said antibody of said immunotoxin conjugate binds to SAS1R on a cancer cell, and wherein the binding of said immunotoxin molecule to the SAS1R molecule on a cell results in the killing, sloughing off or inhibiting of proliferation of the SAS1R expressing cell.

10. The method of claim 9, wherein the cytotoxic agent is a Type I ribosome inactivating protein.

11. The method of claim 10, wherein the Type I ribosome inactivating protein is saporin.

12. The method of claim 9, wherein the antibody is monoclonal, polyclonal, chimeric, human, or humanized.

13. The method of claim 9, wherein the antibody binding fragment is F(ab′)₂, F(ab)₂, Fab′, or Fab.

14. The method of claim 9, wherein the cancer is selected from the group consisting of carcinoma (e.g, head and neck squamous cell carcinoma), sarcoma, uterine cancer, ovarian cancer, lung cancer, adenocarcinoma, adenocarcinoma of the lung, squamous carcinoma, squamous carcinoma of the lung, malignant mixed mullerian tumor, leukemia, lymphoma, neuroblastoma, melanoma, breast cancer, prostate cancer, pancreatic cancer, kidney cancer, bladder and endometrioid carcinoma.

15. A method treat cancer, comprising administering a therapeutically effective dose of a first antibody, or fragment thereof, which binds to SAS1R on a cancer cell, and a second antibody that binds to the first antibody, wherein the second antibody is conjugated to a cytotoxic agent.

16. The method of claim 15, wherein the antibody is monoclonal, polyclonal, chimeric, human, or humanized.

17. The method of claim 15, wherein the antibody binding fragment is F(ab′)₂, F(ab)₂, Fab′, or Fab.

18. The method of claim 15, wherein the cytotoxic agent is a Type I ribosome inactivating protein.

19. The method of claim 18, wherein the Type I ribosome inactivating protein is saporin.

20. A method to diagnose ovarian cancer, uterine cancer, bladder cancer, kidney cancer, pancreatic cancer or head and neck squamous cell carcinoma (HNSCC) comprising measuring SAS1B in a biological sample from ovary, uterus, bladder, kidney, pancreas or head and neck of a subject, and diagnosing ovarian cancer, uterine cancer, bladder cancer, kidney cancer, pancreatic cancer or head and neck squamous cell carcinoma (HNSCC) in said subject based on the presence of SAS1B in the biological sample from ovary, uterus, bladder, kidney, pancreas or head and neck of the subject.

21. The method of claim 20, wherein the subject is mammalian.

22. The method of claim 20, wherein the SAS1B is SAS1B protein, miRNA or mRNA.

23. The method of claim 20, wherein said detection comprises analyzing the results with an analytical device and program.

24. The method of claim 23, wherein the analytical device comprises a computer.

25. The method of claim 24, wherein the analytical device comprise a sequence analyzer.

26. The method of claim 23, wherein the level of SAS1B protein, miRNA or mRNA is quantified with an analytical device and program.

27. The method of claim 20, wherein said SAS1B protein, miRNA, or mRNA is detected using a method selected from the group consisting of ELISA, immunoassay, immunofluorescence, immunohistochemistry, immunoprecipitation, northern blot, western blot, PCR, mass spectrometry, and surface Plasmon resonance.

28. The method of claim 20, wherein the sample is tissue biopsy.
